(19) United States
(12) Patent Application Publication
(10) Pub. No.: US 2026/0345031 A1
(43) Pub. Date: Nov. 5, 2026

(54) SYSTEM AND METHOD FOR MONITORING A PATIENT IN A PATIENT SUPPORT APPARATUS USING A CONTACTLESS SENSING DEVICE

(71) Applicant: UMANO MEDICAL INC., L'Islet (CA)

(72) Inventors: Anouer KEBIR, Levis (CA); Jean-Francois GROU, Montreal (CA); Esther BERTHELOT, Levis (CA); Jerome MARCOTTE, Levis (CA); David MAYEN-MORENO, Levis (CA); Ghislain DEMERS, Beaumont (CA); Jade CLOUATRE, Quebec (CA)

(73) Assignee: UMANO MEDICAL INC., L'Islet, QC (CA)

(21) Appl. No.: 19/109,163

(22) PCT Filed: Sep. 7, 2023
(86) PCT No.: PCT/IB2023/058874

§ 371 Date: Mar. 6, 2025

Related U.S. Application Data
(60) Provisional application No. 63/404,389, filed on Sep. 7, 2022.

(51) Int. Cl. A61B 5/11; A61B 5/00; A61B 5/08

(52) U.S. Cl. CPC A61B 5/1115; A61B 5/0816; A61B 5/1114; A61B 5/7267

(57) ABSTRACT

A system comprises a patient support apparatus and at least one contactless sensing device in proximity. The patient support apparatus includes a patient platform having: a plurality of body panels including a backrest, a frame, the patient platform being mounted to the frame, and the at least one contactless sensing device configured for monitoring at least one vital sign of the patient. The at least one vital sign includes for example a heart rate and a respiratory rate. In some implementations, the at least one sensing device is configured to determine a movement index of the patient. The movement index is used to detect movements made by the patient using a trained machine learning model. The movement index is used to determine a mobility level of the patient. A method of determining the mobility level is also provided.

### CROSS-REFERENCE

The present application claims priority from U.S. Provisional Patent Application Ser. No. 63/404,389 filed on Sep. 7, 2022.

### TECHNICAL FIELD

The technology relates to systems and methods for monitoring a patient in a patient support apparatus such as a hospital bed, and in particular embodiments, to systems, patient support apparatuses and methods for monitoring the vital signs and determining a mobility index or level of a patient in a patient support apparatus.

### BACKGROUND OF THE ART

Many hospital patients need to have their condition monitored on an ongoing basis, including monitoring vital signs like their breathing and heart rate. To improve the comfort of the patient, it is desirable to minimize the number of sensors and other equipment that must be coupled to the patient's body for this purpose. It is also important to have a consistent and reliable system of monitoring the patient's vital signs.

U.S. Patent Application Publication No. 2018/0256082, to Steinberg et al. discloses a system for non-invasively detecting vital signs of a subject, including a) a sub-THz beam source, b) an optical interferometer that is configured to accept the sub-THz beam, split the sub-THz beam into a reference beam and a measurement beam, focus the measurement beam onto a subject, accept a reflection of the beam from the subject and combine the reflection of the measurement beam with the reference beam; c) a detector configured to detect the combined beam; and an electronic circuit configured to receive and analyze the detected combined beam and identify vital signs of the subject.

There is a desire for improved systems, patient support apparatuses and methods of monitoring the vital signs of a patient, determining movements of the patient and a mobility level of the patient.

### SUMMARY

It is an object of the present technology to ameliorate at least one drawback of existing systems.

It is an object of one or more implementations of the present technology to monitor one or more vital signs of a patient in a patient support apparatus using a contactless sensing device, irrespective of one or more of: the position of the patient in the patient support apparatus, the configuration of the patient support apparatus and the location of the patient support apparatus in a room. It is also an object of one or more implementations of the present technology to determine movements of the patient and a mobility index of the patient in the patient support apparatus using the contactless sensing device.

In accordance with one or more embodiments of the present technology, a system comprising at least one contactless sensing device associated with a patient support apparatus is provided to detect and monitor vital signs, movements and a mobility level of a given patient. Depending on the characteristics of the contactless sensing device (e.g., electromagnetic wave emission and reception characteristics including electromagnetic wave frequencies, detection range and field of view), a number, and location(s) of the at least one contactless sensing device relative to the patient support apparatus may be determined to enable detection and monitoring of one or more of the vital signs and movements of the patient, irrespective of anthropometric or physical characteristics of the patient (e.g., weight, size and/or body surface area), irrespective of the position of the patient in the patient support apparatus, the configuration of the patient support apparatus and the location of the patient support apparatus.

In some implementations, the at least one contactless sensing device may be positioned (i.e., fixed at a given location, given position and/or given angle) and/or positionable (i.e., movable between different positions and/or different angles) to cover a patient area of the patient support apparatus (e.g., corresponding to the upper body) such that vital signs are continuously detected when the patient is supported by the patient support apparatus.

In one or more implementations, the number of contactless sensing device may be selected based on the field of detection of the contactless sensing devices to maximize coverage, such that vital signs or movements may be detected when the patient is positioned on the patient support apparatus, irrespectively of the anthropometric or physical characteristics of the patient (e.g., weight, size and/or body surface area). Additionally, or alternatively, the at least one contactless sensing device may be orientable between different positions and/or angles to maximize coverage of the body of the patient. In some implementations, the at least one contactless sensing device may be secured on a structure in proximity to the patient support apparatus, such as a ceiling, a wall, or a pole. In other implementations, the at least one contactless sensing device may be secured to a component of the patient support apparatus or integrated within a component of the patient support apparatus, such as, but not limited to, a panel, a siderail, a headboard and/or a footboard.

In some implementations, the at least one contactless sensing device is a single sensing device coupled to the patient support apparatus to provide an enlarged field of detection. The single sensing device may be coupled to an underside of the back rest and the sensor of the single sensing device may be oriented at an angle towards the head end of the patient support apparatus.

The patient support apparatus may be in the form of a hospital bed, an intensive care unit (ICU) bed, a bariatric bed, a reclining chair, or any other form of support apparatus configured to support at least a portion of a body of a patient without departing from the scope of the present technology.

In accordance with a broad aspect of the present technology, there is provided a system for monitoring at least one vital sign of a patient, the system comprising: a patient support apparatus comprising: a patient platform comprising: a plurality of body panels including a backrest for supporting at least a patient's torso, a frame, the patient platform being mounted to the frame, and at least one contactless sensing device configured to be positioned in proximity to the patient support apparatus to detect at least one vital sign within a respective predetermined field of detection, the at least one contactless sensing device being configured to: transmit electromagnetic signals towards a portion of the body of the patient, receive reflected electromagnetic signals, and detect, based on the transmitted and received reflected electromagnetic signals, at least one vital sign of the patient irrespective of: the patient's anthropomorphic measurements, a patient position within the patient support apparatus, a configuration of the patient support apparatus, and a location of the patient support apparatus.

In one or more implementations of the system, the at least one contactless sensing device is mounted to a respective adjustable positioning mechanism configured to adjust the at least one contactless sensing device to include the patient within the field of detection.

In one or more implementations of the system, the respective adjustable positioning mechanism is configured to adjust the at least one contactless sensing device by performing at least one of: pivoting the at least one contactless sensing device about at least one pivot axis, and positioning the at least one contactless sensing device along at least one translation axis.

In one or more implementations of the system, further comprising a further sensor configured to estimate a patient position, and the respective adjustable positioning mechanism is operatively connected to the further sensor to adjust the plurality of sensing devices based on the patient position.

In one or more implementations of the system, the further sensor comprises at least one of: load cells of the patient support apparatus, pressure sensors of a mattress, and a camera.

In one or more implementations of the system, the at least one contactless sensing device is one of: integrated within the backrest of the patient support apparatus, and mounted to an underside of the backrest of the patient support apparatus.

In one or more implementations of the system, the at least one contactless sensing device is a single contactless sensing device comprising a sensor oriented at a respective angle towards a head end of the patient support apparatus, the respective angle being based on a field of view of the sensor to enlarge the field of detection.

In one or more implementations of the system, the respective angle corresponds to 90 degrees minus the field of view of the sensor in degrees.

In one or more implementations of the system, the at least one contactless sensing device is one of: integrated within a respective siderail of the patient support apparatus, and mounted to a respective siderail of the patient support apparatus.

In one or more implementations of the system, the at least one contactless sensing device is one of: integrated within a headboard of the patient support apparatus, and mounted to a headboard of the patient support apparatus.

In one or more implementations of the system, the at least one contactless sensing device is one of: integrated within a footboard of the patient support apparatus, and mounted to a footboard of the patient support apparatus.

In one or more implementations of the system, the at least one contactless sensing device is mounted above the patient platform on a vertical support secured to the patient support apparatus.

In one or more implementations of the system, the at least one contactless sensing device is mounted to one of a ceiling and a wall.

In one or more implementations of the system, the at least one contactless sensing device comprises a plurality of contactless sensing devices, each of the plurality of contactless sensing devices being positioned at a respective position, the plurality of contactless sensing devices having non-overlapping respective fields of view.

In one or more implementations of the system, the plurality of contactless sensing devices comprise a first contactless sensing device coupled to a first side of the patient support apparatus and a second contactless sensing device coupled to a second side opposite the first side of the patient support apparatus.

In one or more implementations of the system, the at least one contactless sensing device is configured to use radiofrequency (RF) ranges.

In one or more implementations of the system, the at least one contactless sensing device is configured to use one of: millimeter waves (mm Wave) frequencies, ultra-wideband (UWB) frequencies, and Wi-Fi frequencies.

In one or more implementations of the system, the field of detection of the at least one contactless sensing device has a maximum detection range of at least 1.5 meters.

In one or more implementations of the system, the at least one vital sign of the patient comprises at least one of: a heart rate and a respiratory rate.

In one or more implementations of the system, the at least one contactless sensing device is configured to detect movements of the patient based on the transmitted and received reflected electromagnetic signals.

In one or more implementations of the system, the at least one contactless sensing device is configured to detect movements of the patient when the at least one contactless sensing device does not detect the at least one vital sign.

In one or more implementations of the system, the contactless sensing device is configured to determine a movement index indicative of a level of motion of the patient supported by the patient platform.

In one or more implementations of the system, the system comprises a processing unit configured to: receive a set of movement indexes for a given period of time, classify, using a trained machine learning model, each respective movement index of the set of movement indexes during the given period of time as being one of: a respective minor movement and a respective major movement, and determine, based on a number of respective minor movements and a number of respective major movements, a mobility level of the patient during the period of time.

In one or more implementations of the system, the processing unit is configured to use an empirical model to determine the mobility level of the patient during the period of time.

In one or more implementations of the system, the mobility level is based on a Braden scale.

In one or more implementations of the system, the at least one contactless sensing device is configured to use optical frequencies.

In one or more implementations of the system, the at least one contactless sensing device is configured to use infrared frequencies, and the at least one vital sign comprises a body temperature of the patient.

In accordance with a broad aspect of the present technology, there is provided a patient support apparatus comprising: a patient platform comprising: a plurality of body panels including a backrest for supporting at least a patient's torso, a frame, the patient platform being mounted to the frame, and at least one contactless sensing device configured to be coupled to the patient support apparatus to detect at least one vital sign within a predetermined field of detection, the at least one contactless sensing device being configured to: transmit electromagnetic signals towards a portion of the body of the patient, receive reflected electromagnetic signals, and detect, based on the transmitted and received reflected electromagnetic signals, at least one vital sign of the patient within the predetermined field of detection, irrespective of: anthropomorphic measurements of the patient, a patient position within the patient support apparatus, a configuration of the patient support apparatus, and a location of the patient support apparatus.

In one or more implementations of the patient support apparatus, the at least one contactless sensing device is configured to be coupled to the patient support apparatus by being one of: being mounted on a respective component of the patient support apparatus, and integrated to a respective component of the patient support apparatus.

In one or more implementations of the patient support apparatus, the at least one contactless sensing device is mounted to a respective adjustable positioning mechanism configured to adjust the at least one contactless sensing device to include a portion of a body of the patient within the field of detection, the respective adjustable positioning mechanism being coupled to the respective component of the patient support apparatus.

In one or more implementations of the patient support apparatus, the respective adjustable positioning mechanism is configured to adjust the at least one contactless sensing device by performing at least one of: pivoting the at least one contactless sensing device about at least one pivot axis, and positioning the at least one contactless sensing device along at least one translation axis.

In one or more implementations of the patient support apparatus, the patient support apparatus comprises a headboard, a footboard and siderails, and the respective component of the patient support apparatus comprises at least one of headboard, a footboard and siderails.

In one or more implementations of the patient support apparatus, the at least one contactless sensing device is coupled to an underside of the backrest.

In one or more implementations of the patient support apparatus, the contactless sensing device is positioned and oriented towards the patient's lower back.

In one or more implementations of the patient support apparatus, the at least one contactless sensing device is a single contactless sensing device comprising a sensor oriented at a respective angle towards a head end of the patient support apparatus, the respective angle being based on a field of view of the sensor to enlarge the field of detection.

In one or more implementations of the patient support apparatus, the respective angle corresponds to 90 minus the field of view of the sensor in degrees.

In one or more implementations of the patient support apparatus, the at least one contactless sensing device comprises a plurality of contactless sensing devices, each of the plurality of contactless sensing devices being positioned at a respective position, the plurality of contactless sensing devices having non-overlapping respective fields of detection within the patient platform of the patient support apparatus.

In one or more implementations of the patient support apparatus, the plurality of contactless sensing devices comprises a first contactless sensing device coupled to a first side of the patient support apparatus and a second contactless sensing device coupled to a second side opposite the first side of the patient support apparatus.

In one or more implementations of the patient support apparatus, the at least one contactless sensing device is configured to use radiofrequency (RF) ranges.

In one or more implementations of the patient support apparatus, the at least one contactless sensing device is configured to use one of: millimeter waves (mmWave), ultra-wideband (UWB), and Wi-Fi frequencies.

In one or more implementations of the patient support apparatus, the field of detection of the at least one contactless sensing device has a maximum detection range of at least 1.5 m.

In one or more implementations of the patient support apparatus, the at least one vital sign of the patient comprises at least one of: a heart rate and a respiratory rate.

In one or more implementations of the patient support apparatus, the at least one contactless sensing device is configured to detect movements of the patient based on the transmitted and received reflected electromagnetic signals.

In one or more implementations of the patient support apparatus, the at least one contactless sensing device is configured to detect movements of the patient when the at least one contactless sensing device does not detect the at least one vital sign.

In one or more implementations of the patient support apparatus, the contactless sensing device is configured to determine a movement index indicative of a level of motion of the patient supported by the patient platform.

In one or more implementations of the patient support apparatus, a processing unit is configured to a processing unit is configured to: receive a set of movement indexes for a given period of time, classify, using a trained machine learning model, each respective movement index of the set of movement indexes during the given period of time as being one of: a respective minor movement and a respective major movement, and determine, based on a number of respective minor movements and a number of respective major movements, a mobility level of the patient during the period of time.

In one or more implementations of the patient support apparatus, the processing unit is configured to use an empirical model to determine the mobility level of the patient during the period of time.

In one or more implementations of the patient support apparatus, the mobility level is based on a Braden scale.

In accordance with a broad aspect of the present technology, there is provided a method for determining a mobility of a patient supported a patient support apparatus, the patient support apparatus being associated with a contactless sensing device configured to detect vital signs of a patient, the method being executed by at least one processing unit, the method comprising: receiving, from the sensing device, a set of movement indexes indicative of a movement of the patient during a period of time, classifying, using a trained machine learning model, each respective movement index of the set of movement indexes during the period of time as being one of: a respective minor movement and a respective major movement, and determining, based on a respective number of minor movements and a respective number of major movements, a mobility level of the patient during the period of time.

In one or more implementations of the method, further comprising: receiving a maximal amplitude of the set of movement indexes and a sum of the movement indexes, said classifying, using the trained machine learning model, each respective movement index of the set of movement indexes during the period of time as being one of: the respective minor movement and the respective major movement is further based on the maximal amplitude of the set of movement indexes and a sum of the movement indexes.

In one or more implementations of the method, the at least one processing unit is configured to use an empirical model to determine the mobility level of the patient during the period of time.

In one or more implementations of the method, the mobility level is based on a Braden scale.

In one or more implementations of the method, the method further comprises: detecting, by the contactless sensing device, before the given period of time, at least one vital sign of the patient.

In one or more implementations of the method, the at least one vital sign comprises at least one of a heart rate (HR) and a respiratory rate (RR).

In one or more implementations of the method, the method further comprises: transmitting an indication of the mobility level of the patient to a computing device of a medical professional.

According to another aspect, there is provided a hospital bed comprising: a patient platform comprising: a plurality of body panels including a backrest for supporting at least a patient's torso; a frame, the patient platform being mounted to the frame; and a plurality of sensing devices for monitoring at least one vital sign of the patient, the sensing devices having non-overlapping fields of detection within the patient platform of the bed.

Optionally, in any of the previous aspects, the plurality of sensing devices include at least one first sensing device on a first side of the bed and at least one second sensing device on a second side of the bed.

Optionally, in any of the previous aspects, the second side of the bed is opposite the first side of the bed.

Optionally, in any of the previous aspects, the plurality of sensing devices include first and second sensing devices disposed on the backrest.

Optionally, in any of the previous aspects, the plurality of sensing devices are disposed on an underside of the backrest.

Optionally, in any of the previous aspects, the backrest comprises a material that is substantially transparent to a wavelength used by the plurality of sensing devices.

Optionally, in any of the previous aspects, the first and second sensing devices are coupled to a support structure mounted to the backrest.

Optionally, in any of the previous aspects, the plurality of sensing devices include a first sensing device disposed in or on a first siderail of the hospital bed and a second sensing device disposed in or on a second siderail of the hospital bed.

Optionally, in any of the previous aspects, the first and second siderails comprise a material that is substantially transparent to a wavelength used by the plurality of sensing devices.

Optionally, in any of the previous aspects, the plurality of sensing devices include first and second sensing devices disposed in or on a first siderail of the hospital bed.

Optionally, in any of the previous aspects, the first siderail comprises a material that is substantially transparent to a wavelength used by the plurality of sensing devices.

Optionally, in any of the previous aspects, the plurality of sensing devices include first and second sensing devices disposed in or on the footboard.

Optionally, in any of the previous aspects, the footboard comprises a material that is substantially transparent to a wavelength used by the plurality of sensing devices.

Optionally, in any of the previous aspects, the plurality of sensing devices are mounted above the patient platform on a vertical support coupled to the bed.

Optionally, in any of the previous aspects, the bed is configured to communicate patient position information to the plurality of sensing devices; and wherein the plurality of sensing devices are configured to orient the plurality of sensing devices based on the patient position information.

Optionally, in any of the previous aspects, the sensing devices monitor the at least one vital sign of the patient by using one of: millimeter waves, radar, visible light, invisible light, or ultra-wideband (UWB).

Optionally, in any of the previous aspects, the at least one vital sign of the patient includes one or more of: heart rate, interval and variability; respiratory rate and depth; inhale-exhale ratio; biometric identification; lying position and movement; sleep apnea detection; fall risk detection; and bed exit frequency.

According to a another broad aspect, there is provided a hospital bed comprising: a patient platform comprising: a plurality of body panels including a backrest for supporting at least a patient's torso; a frame, the patient platform being mounted to the frame; and at least one sensing device for monitoring at least one vital sign of the patient, the at least one sensing device being movable with respect to the patient platform.

Optionally, in any of the previous aspects, the at least one sensing device is movable by being pivotable about at least one axis.

Optionally, in any of the previous aspects, the at least one sensing device is disposed on an underside of the backrest; and the at least one sensing device is movable by being translatable in at least one direction.

Optionally, in any of the previous aspects, the backrest comprises a material that is substantially transparent to a wavelength used by the plurality of sensing devices.

Optionally, in any of the previous aspects, the at least one sensing device is coupled to a support structure mounted to the backrest.

Optionally, in any of the previous aspects, the at least one sensing device is disposed in or on a siderail; and wherein the at least one sensing device is movable by being translatable in at least one direction.

Optionally, in any of the previous aspects, the siderail comprises a material that is substantially transparent to a wavelength used by the plurality of sensing devices.

Optionally, in any of the previous aspects, the at least one sensing device is disposed in or on the footboard; and wherein the at least one sensing device is movable by being translatable in at least one direction.

Optionally, in any of the previous aspects, the footboard comprises a material that is substantially transparent to a wavelength used by the plurality of sensing devices.

Optionally, in any of the previous aspects, the at least one sensing device is supported above the patient platform by on a vertical support coupled to the bed.

Optionally, in any of the previous aspects, the hospital bed further includes at least one sensor for determining a position of a patient within the hospital bed, the at least one sensing device being movable in response to the determination.

Optionally, in any of the previous aspects, the at least one sensing device monitors the at least one vital sign of the patient by using one of: millimeter waves, radar, visible light, invisible light, or ultra-wideband (UWB).

Optionally, in any of the previous aspects, the at least one vital sign of the patient includes one or more of: heart rate, interval and variability; respiratory rate and depth; inhale-exhale ratio; biometric identification; lying position and movement; sleep apnea detection; fall risk detection; and bed exit frequency.

According to a another broad aspect, there is provided a hospital bed comprising: a patient platform comprising: a plurality of body panels including a backrest for supporting at least a patient's torso; a frame, the patient platform being mounted to the frame; and at least one sensing device for monitoring at least one vital sign of the patient.

Optionally, in any of the previous aspects, the sensing device disposed in or on a siderail of the hospital bed.

Optionally, in any of the previous aspects, the siderail comprises a material that is substantially transparent to a wavelength used by the plurality of sensing devices.

Optionally, in any of the previous aspects, the plurality of sensing devices include first and second sensing devices disposed on the backrest.

Optionally, in any of the previous aspects, the sensing device is disposed on an underside (i.e., bottom surface) of the backrest.

Optionally, in any of the previous aspects, the backrest comprises a material that is substantially transparent to a wavelength used by the sensing device.

Optionally, in any of the previous aspects, the at least one sensing device is coupled to a support structure mounted to the backrest.

Optionally, in any of the previous aspects, the sensing device disposed in or on the footboard.

Optionally, in any of the previous aspects, the footboard comprises a material that is substantially transparent to a wavelength used by the plurality of sensing devices.

Optionally, in any of the previous aspects, the sensing device is mounted above the patient platform on a vertical support coupled to the bed.

Optionally, in any of the previous aspects, the sensing devices monitor the at least one vital sign of the patient by using one of: millimeter waves, radar, visible light, invisible light, or ultra-wideband (UWB).

Optionally, in any of the previous aspects, the at least one vital sign of the patient includes one or more of: heart rate, interval and variability; respiratory rate and depth; inhale-exhale ratio; biometric identification; lying position and movement; sleep apnea detection; fall risk detection; and bed exit frequency.

According to a another broad aspect, there is provided an apparatus for monitoring at least one vital sign of a patient, the apparatus comprising: a housing mounted on one of a wall or a ceiling of a room; at least one sensing device disposed in the housing; and a receiver for receiving a signal from a hospital bed, the signal being indicative of a position of a patient in the hospital bed; the apparatus being responsive to the received signal to orient the at least one sensing device in the direction of the patient.

Optionally, in any of the previous aspects, the housing is configured to rotate about at least one axis; and wherein orienting the at least one sensing device in the direction of the patient comprises rotating the housing about the at least one axis.

Optionally, in any of the previous aspects, the housing is configured to move translationally along the wall or the ceiling; and wherein orienting the at least one sensing device in the direction of the patient comprises translating the housing along the wall or the ceiling.

According to another broad aspect, there is provided a method of operating a sensing device, comprising: receiving, from a hospital bed, a first signal indicative of a position of a patient within a patient area of the hospital bed; orienting the sensing device based on the received first signal; and determining, by the sensing device, information indicative of at least one vital sign of the patient.

Optionally, in any of the previous aspects, the method further includes: transmitting, by the sensing device, a second signal to the hospital bed, the second signal confirming that the sensing device has received the first signal.

Optionally, in any of the previous aspects, the method further includes: transmitting, by the sensing device, a third signal to the hospital bed, the third signal containing the information indicative of the at least one vital sign of the patient.

### BRIEF DESCRIPTION OF THE DRAWINGS

Having thus generally described the nature of the technology, reference will now be made to the accompanying drawings, showing by way of illustration example embodiments thereof and in which:

FIG. 1 is a perspective view of a hospital bed to which the present technology may be applied;

FIG. 2 is a schematic illustration of a sensing device that can be used along with the present technology;

FIG. 3 is a perspective view of a hospital bed according to a first embodiment;

FIG. 4 is a perspective view of a hospital bed according to a second embodiment;

FIG. 5 is a perspective view of a hospital bed according to a third embodiment;

FIG. 6 is a perspective view of a hospital bed according to a fourth embodiment;

FIG. 7 is a perspective view of a hospital bed according to a fifth embodiment;

FIG. 8 is a perspective view of a hospital bed according to a sixth embodiment;

FIG. 9 is a perspective view of a hospital bed according to a seventh embodiment;

FIG. 10 is a perspective view of a hospital bed according to an eighth embodiment;

FIG. 11 is a perspective view of a hospital bed according to a ninth embodiment;

FIG. 12 is a perspective view of a hospital bed according to a tenth embodiment;

FIG. 13 is a perspective view of a hospital bed according to an eleventh embodiment;

FIG. 14 is a perspective view of a hospital bed according to a twelfth embodiment;

FIG. 15 is a perspective view of a hospital bed according to a thirteenth embodiment;

FIG. 16 is a perspective view of a hospital bed according to a fourteenth embodiment;

FIG. 17 is a call flow diagram of a method of orienting a sensing device according to an embodiment;

FIG. 18 is a perspective view of a hospital bed with a sensing device taken from the top right, according to a fifteenth embodiment of the present technology;

FIG. 19 is a perspective view of the hospital bed with the sensing device of FIG. 18 taken from the top left;

FIG. 20 is an enlarged view of the sensing device of the hospital bed of FIG. 18;

FIG. 21 is a cross-section of the sensing device taken along line AA′ of FIG. 20;

FIG. 22 is a view of a portion of a body of a patient targeted by the sensing device for detecting vital signs in accordance with one or more non-limiting embodiments of the present technology;

FIG. 23A and FIG. 23B are schematic diagrams illustrating the field of view of the sensing device at an angle of 0 degree relative to the patient and at an angle of 25 degrees relative to the patient, respectively;

FIG. 24A and FIG. 24B are schematic diagrams illustrating the field of view of the sensing device taken from the right side and from the top, respectively;

FIG. 25 is a schematic diagram of an environment and system comprising the hospital bed and the sensing device in accordance with one or more non-limiting embodiments of the present technology;

FIG. 26 is a schematic diagram of a patient mobility determination procedure in accordance with one or more non-limiting embodiments of the present technology; and

FIG. 27 is a flow chart of a method of estimating a mobility index of a patient, the method being illustrated in accordance with one or more non-limiting embodiments of the present technology.

### DETAILED DESCRIPTION

In the present description, all references to the position of a part of a patient support apparatus or other equipment are intended to refer to a normal lying or sleeping position of the patient in the bed, unless indicated otherwise.

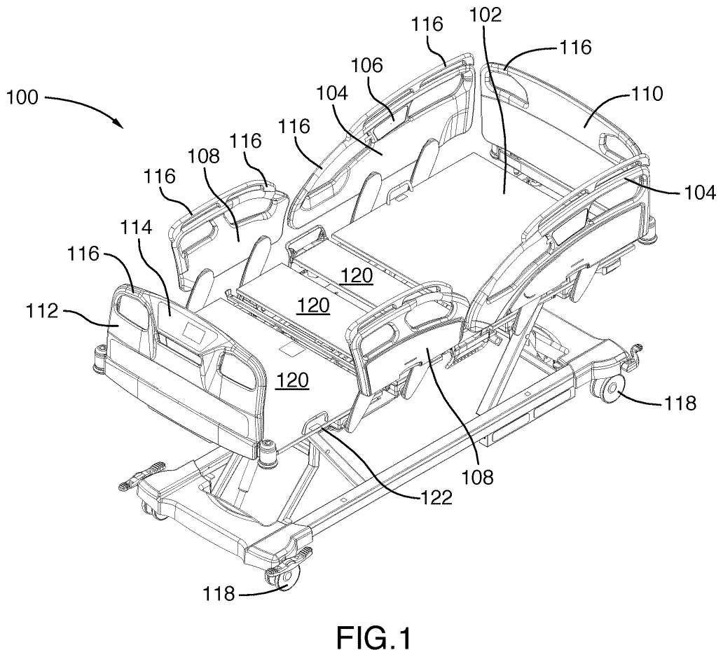

Referring to FIG. 1, a patient support apparatus implemented as a hospital bed 100 will be described.

Some of the structural components of the hospital bed 100 will be designated hereinafter as “right”, “left”, “head” and “foot” from the reference point of an individual lying on his/her back on the support surface of the mattress provided on the hospital bed 100 with his/her head oriented toward the head end of the hospital bed 100 and his/her feet oriented toward the foot end of the hospital bed 100.

A backrest 102 supports the head and torso of the patient. A pair of siderails 104 at the head end of the bed are intended to prevent the patient from falling out of the bed. The angle of the backrest 102 can typically be raised or lowered, for example using a control panel 106 provided on the siderails 104. The siderails 104 can typically be raised or lowered together with the backrest 102. Siderails 108 are provided near the middle or foot of the hospital bed 100. The siderails 108 can be lowered so as not to obstruct the patient when entering or exiting the hospital bed 100.

A headboard 110 is located at the head of the hospital bed 100 and a footboard 112 is located at the foot end of the hospital bed 100. The footboard 112 may have a control panel 114 for controlling one or more functions of the hospital bed 100. Handles 116 are provided in multiple locations, such as on the headboard 110, footboard 112, and siderails 104, 108, to facilitate moving the hospital bed 100 from one place to another using the wheels 118. In some embodiments, the footboard 112 may be a separate module that can be removed and replaced, for example to upgrade to a newer footboard having additional features. Additional body panels 120 support the lower portion of the patient's body that is not supported by the backrest 102. All of the body panels 120, including the backrest 102, define a patient platform that supports the patient within a patient area of the hospital bed 100. The various parts of the hospital bed 100 are mounted to and supported by a frame 122.

The hospital bed 100 may have additional features, such as the ability to accommodate the patient in a seated position by moving one or more of the body panels 120, including the backrest 102. In such embodiments, the hospital bed 100 includes a plurality of pivoting systems including one or more actuators for pivoting one or more of the backrest 102, the lower body support panel and the core support panels relative to the frame 122. In some implementations of the present technology, the hospital bed 100 is designed to accommodate a variety of medical requirements and patient preferences through multiple configurable positions. The hospital bed 100 may be adjusted into the following configurations: (i) a flat position suitable for standard patient rest and post-operative care; (ii) a Fowler's position, in which the head section is elevated at an angle between 30 to 90 degrees to aid in respiratory function and facilitate patient interaction; (iii) a Trendelenburg position that tilts the bed so the patient's head is positioned lower than the feet, commonly employed for certain surgical procedures and medical treatments; (iv) a Reverse Trendelenburg position, where the head is elevated higher than the feet, often used to enhance circulation and alleviate pressure on the lower back; and (v) a lateral tilt configuration that allows for side-to-side angulation of the bed for pressure relief and postural drainage. The hospital bed 100 configurations are actuated via a control mechanism (e.g., pivoting systems), which may be either manual or electronic. It will be appreciated that other configurations of the hospital bed 100 may be possible without departing from the scope of the present disclosure.

The hospital bed 100 may further include a plurality of sensors for sensing different parameters of the hospital bed 100, such as bed configuration (e.g., angles and positions of the backrest, lower body and core support panels, height of the elevation system, etc.), or for sensing parameters related to the patient (patient weight, location of the patient, etc.).

The hospital bed 100 includes a control unit 180 or controller 180 operatively connected to inter alia an elevation system (not shown), the plurality of pivoting systems (not numbered) and the plurality of sensors (not shown) configured to receive and transmit signals thereto. The controller 180 may be operatively connected to the components of the hospital bed 100 via a circuitry (not shown). The controller 180 is used to control various functions of the hospital bed 100. In one embodiment, the controller 180 is mounted on the patient support assembly, for example below one of the panels of the patient support surface.

The hospital bed 100 includes one or more control panels 106, 114 (or input/output interface 106) operatively connected to the controller 180. The one or more control panels 106, 114 may be integrated into the footboard 124, into the headboard 110 or into one or more of the siderails 104, 108. Alternatively, the control panels 106, 114 may be provided as a separate unit located near the hospital bed 100 or even at a location remote from the hospital bed 100 and operatively connected to the hospital bed 100. The one or more control panels 106, 114 is operatively connected to the controller 180 from which it receives and transmits signals, such as signals from sensor(s), actuators, and other mechanisms on the hospital bed 100. The one or more control panels 106, 114 may for example display sensed parameters and configuration parameters and allow a user to transmit commands to the controller 180 for controlling various functions of the hospital bed 100, such as actuating the actuators for pivoting panels, controlling the elevation system and the like. As a non-limiting example, the one or more control panels 106, 114 may be implemented as one or more of a touchscreen, as a digital screen with physical input buttons or touch pads, or a combination thereof.

The controller 180 comprises one or more processing unit(s), one or more memories, one or more transceivers, input/output interfaces and communication interfaces (not shown). It will be appreciated that the controller 180 is an embodiment of a computing device.

The one or more communication interfaces may include wired and wireless communication interfaces to connect components of the hospital bed 100 to other medical devices, computing devices (e.g., nurse station computer, server(s) and mobile devices), and communication networks (e.g., hospital network) to transmit and receive data.

The one or more communication modules may include wireless communication modules configured to transmit and receive one or more types of wireless signals (e.g., Wi-Fi, Bluetooth™ (including Bluetooth Low Energy (BLE)), Zigbee, cellular/GSM, Infrared (IR), radiofrequency (RF), ultrawide band (UWB), Near Field Communication (NFC), Radio-Frequency Identification (RFID), etc.) and wired communication modules (e.g., ethernet, USB, Serial, fiber optics, etc.). The hospital bed 100 may optionally perform monitoring functions. For example, weight sensors (not shown) in a plurality of locations under the mattress (not shown) can be used to monitor for patient movement, as well as to determine the patient's location within the hospital bed 100 or whether the patient has left the hospital bed 100. In one example, the weight sensors may be integrated into the wheels of the hospital bed 100. In another example, one or more pressure sensors, such as pressure-sensitive mats sold under the Velostat™ name, may be disposed on one or more body panels 120 under the patient area of the hospital bed 100. In another example, the location of the patient can be determined by the use of a camera (not shown), for example an infrared (IR) or visible light camera or other type of sensor, mounted in any convenient location in the room and directed at the patient area of the bed.

The one or more communication modules of the hospital bed 100 may be configured for communicating with other sensors that may or may not be attached to the patient, and may be configured for communicating sensor information to computing devices in the hospital so that it can be accessed by medical personnel. The hospital bed 100 may have additional features and capabilities, which may vary depending on the particular model of bed.

A mattress (not shown) is typically provided and removably attached to the hospital bed 100.

Non-limiting examples of implementations of the hospital bed 100 include ook snow, ook snow ALL, ook snow MH, ook cocoon, and the Max-Secure platform bed manufactured by Umano Medical Inc. of Quebec, Canada.

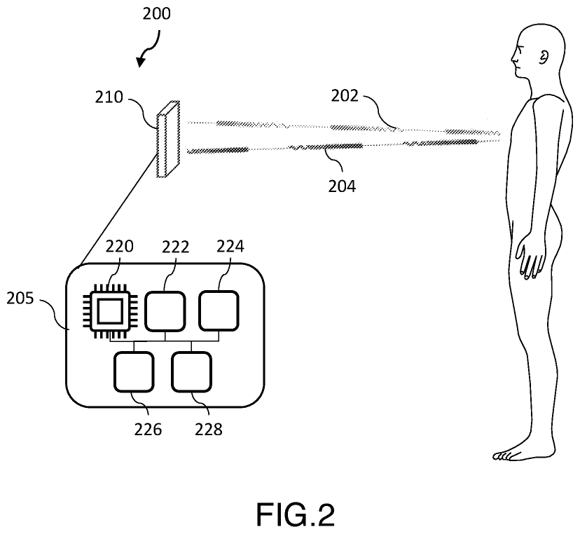

Referring now to FIG. 2, a contactless sensing device 200 is configured to detect one or more vital signs of a given patient 280. The contactless sensing device 200, which is also referred the sensing device 200, is configured to detect the one or more vital signs in a contactless manner.

In the context of the present disclosure, a “contactless sensing device” refers to an electronic sensor specifically designed to detect, measure, and monitor various parameters or conditions without requiring physical contact with the target object or subject by using electromagnetic waves or signals.

The sensing device 200 is configured to inter alia: (i) transmit electromagnetic signal(s) towards the body of a patient; (ii) receive reflected electromagnetic signal(s) from a portion of the body of the patient; (iii) detect one or more vital signs of the patient using the transmitted and reflected electromagnetic signals; (iv) detect movements of the patient using the transmitted and reflected electromagnetic signals.

The one or more vital signs of the patient include heart rate (HR) and respiratory rate (RR). In some implementations, the one or more vital signs may further comprise heart interval and variability (HRV); respiratory rate and depth; inhale-exhale ratio; biometric identification; lying position and movement; sleep apnea detection; fall risk detection; and bed exit frequency.

In one or more implementations, the sensing device 200 enables detecting one or more vital signs of the given patient 280 and movements of the given patient without human intervention and independently from characteristics of the given patient 280 (e.g., size, weight, position), the configuration of the bed 100 (e.g., adjustable positions), and the location of the bed 100 (e.g., location in a room). As a non-limiting example, the sensing device 200 may detect vital signs and movement of the patient 280 irrespective of the patient posture, the patient position and/or bed configurations, which may include supine position, semi-sitting position (i.e., Fowler's position), orthopneic, prone, lateral, semiprone (i.e., Sim's position), lithotomy, Trendelenburg's, reverse Trendelenburg, knee-chest position, jackknife position, and kidney position.

In some embodiments of the present technology, such as when the sensing device 200 is disposed within or under a panel of the bed 100, the sensing device 200 enables detecting the one or more vital signs through different types of mattresses, including air mattresses when not used in therapy mode (e.g., lateral rotation, alternating pressure, pulsation, etc.).

The contactless nature of the sensing device 200 enables inter alia: (i) a reduced risk of skin irritation or pressure sores commonly associated with contact-based sensors; (ii) ease of use, as it eliminates the need for frequent manual adjustments by healthcare professionals; (iii) enhanced patient comfort, as there is no physical contact with monitoring equipment; and (iv) continuous monitoring without disturbing the patient's rest or movement. The use of the contactless sensing device 200 thereby improves the overall quality of patient care while streamlining the workflow for healthcare providers.

In addition to detection and monitoring of one or more vital signs of the given patient, the sensing device 200 is configured to detect movements and assessing mobility of the given patient, and transmitting an indication of a deterioration of the vital signs of the patient and/or a change in movement and mobility. As a non-limiting example, the indication may be transmitted to an internet of things (IOT) device or a computing device, such as a nurse station computer or a mobile device of medical personnel. Additionally, or alternatively, the indication may also be transmitted in the form of a command signal to other connected devices or to control systems of the hospital bed 100 to cause execution of an action.

The sensing device 200 comprises a housing 210 and a sensor 205 disposed within the housing 210. The housing 210 provides support and protection to the components of the sensing device 200 and enables securing the sensing device 200 to structures and/or objects, as will be explained below.

The sensor 205 comprises one or more processing units 220, one or more memories 222, one or more transceivers 224, input/output interfaces 226 and communication interfaces 228. The sensing device 200 may also comprise additional wired or wireless communication interfaces 228 operable to transmit and receive signals to computing devices. In alternative implementations, the sensing device 200 further comprises input/output (I/O) interfaces including user interface(s), such as a screen, a keyboard, button, a touchscreen. In the following, the terms “sensing device” and “sensor” may be used interchangeably unless provided otherwise.

The hardware components of the sensing device 200, including the one or more processing units 220, the one or more memories 222, transceiver(s) 224, the communication interface(s) 228, and the input/output interface(s) may be replaced with alternatives. In one or more implementations, the one or more processing units 220 may include a single-core microprocessor. In one or more other implementations, the one or more processing units 220 may include a multi-core microprocessor. In one or more alternative implementations, the one or more processing units may include a microcontroller, a digital signal processor (DSP), an integrated circuit purposed for specific operations within an embedded system, or a Field-Programmable Gate Array (FPGA) or Application-Specific Integrated Circuit (ASIC).

The one or more memories 222 may include volatile and non-volatile memories. In one or more implementations, the one or more memories may include volatile memory, such as Random Access Memory (RAM), and/or alternatively Static Random Access Memory (SRAM) or Dynamic Random Access Memory (DRAM). In one or more implementations, the one or more memories may include non-volatile memory, such as Flash memory and/or alternatively Electrically Erasable Programmable Read-Only Memory (EEPROM) or Ferroelectric RAM (FRAM).

In one or more implementations, the sensor 205 may be implemented as an integrated circuit (IC) such as a system on a chip (SoC). As a non-limiting example, the one or more processing units 220 and memories 222 may be implemented as an ARM® Cortex® M7 MCU chip licensed by ARM from Cambridge, England, UK.

The one or more transceivers 224 comprise transmission and reception antennas with associated electronic circuitry. The one or more transceivers 224 may have a dedicated control unit or may be controlled by the one or more processing units 220 of the sensor 205. The one or more transceivers 224 comprise transmitter antennas configured to transmit one or more electromagnetic signals and receiver antennas configured to receive incoming electromagnetic signals. The electronic circuitry and/or the processing units 220 are configured to convert input signals into electromagnetic waves and convert back the electromagnetic waves into signals.

The sensing device 200 is configured to transmit, via the one or more transceivers 224, electromagnetic signals towards a target and receive, via the one or more transceivers 224, electromagnetic signals reflected from the target. The target includes at least a portion of a body of a patient supported by a patient support apparatus.

In one or more implementations, the one or more transceivers 224 are configured to use one of: (i) millimeter waves (mmWave) (wavelengths from 10 mm to 1 mm and frequencies between 30 to 300 GHz.; (ii) ultrawideband (UWB) (frequencies between 3.1 to 10.6 GHz); and (iii) Wi-Fi (e.g., 802.11n Wi-Fi standard, frequencies in 2.4 GHz and 5 GHz bands). It will be appreciated that other frequencies in the radiofrequency (RF) domain may be used.

In one or more implementations, depending on the type of transmitted signals, the one or more transceivers 224 are configured to use radar technology including continuous wave (CW), pulsed, frequency-modulated continuous wave (FMCW) and stepped-frequency continuous-wave (SFCW).

In one or more other implementations, the sensing device 200 may be configured to use photonic radars (Ka-band (26.5-40 GHz)) technologies, light detection and ranging (LiDAR) technologies, infrared frequencies (e.g., near-infrared (NIR), short-wave infrared (SWIR), /thermal imaging and the like. It will be appreciated that in such implementations, the sensing device 200 should be positioned to allow for unobstructed measurements of vital signs, minimizing signal interference. In the embodiments where the sensing device 200 is configured to use NIR, vital signs such as pulse oximetry may be detected. In embodiments where the sensing device 200 is configured to use LWIR, vital signs such as skin temperature may be detected. It will be appreciated that a sensing device implemented as a photonics radar, LIDAR, infrared and thermal imaging may be used in combination with sensing devices 200 using radiofrequencies (e.g., mmWave, UWB, and Wi-Fi).

In some implementations, the sensing device 200 uses impulse radio ultrawideband (UWB) technology to transmit and receive pulses, such as sub-nanosecond pulses without carriers or modulated short pulses with carriers. In one or more other implementations, the sensing device 200 uses frequency modulation continuous wave (FMCW) radar.

The sensing device 200 is configured to process the electromagnetic signal(s) transmitted and received by one or more transceivers 224 to extract relevant information such as one or more vital signs and mobility of the patient.

The sensing device 200 may be operable to cause transmission of electromagnetic signals 202 towards a patient and to receive reflected signals 204. The sensing device 200 is operable to compare the reflected signals 204 to the transmitted signals 202 using signal processing techniques to detect the distance and motion of objects. In some implementations, the sensing device 200 enables detecting one or more vital signs of a patient based on the change in the signals due to displacement caused by human lungs, heart during respiration and heart beating.

To achieve that purpose, the sensing device 200 is configured to use digital signal processing techniques and algorithms. The signal processing techniques and algorithms used depend on the type and frequency of the electromagnetic waves used by the sensing device 200. For example, impulse radio UWB radar uses pulses to measure range, and chest motion can be measured by the amplitude, and frequency modulation continuous wave (FMCW) radar uses chirp to measure the distance and velocity, and vital signs can be estimated with phase information. It will be appreciated that the signal processing may be executed by the processing unit of the sensing device 200 and/or by another processing unit.

Non-limiting examples of signal processing techniques include fast Fourier transform (FFT), Ensemble empirical mode decomposition (EEMD) and Continuous wavelet transforms (CWT), Wavelet transform, MTI and Chirp Z-transform (CZT), Multiple Higher Order Cumulant (MHOC), Spectrum-Averaged Harmonic Path (SHAPA) and the like.

A non-limiting example of a suitable sensing device for one or more embodiments of the present technology is manufactured by Neteera Technologies Ltd. from Jerusalem, Israel. Another non-limiting example of a suitable sensing device 200 for one or more other embodiments of the present technology is manufactured by Xandar Kardian Inc. from Seoul, South Korea. It is contemplated that other types of sensing devices might be developed or used to determine vital signs and information about the patient that could be of interest to medical practitioners, such as one or more of: heart rate, interval and variability; respiratory rate and depth; inhale-exhale ratio; biometric identification; lying position and movement; sleep apnea detection; fall risk detection; and bed exit frequency.

It will be appreciated that the use of contactless sensing devices (e.g., sensing device 200) may present a number of challenges in a hospital setting. Many patients require continual monitoring of their heart rate and breathing rate, in particular while the patient is in a hospital bed. In some implementations, the sensing device 200 may have a relatively narrow field of view, for example about 20 degrees, and may be only sensitive to objects within a specific distance range, for example from about 20 cm (8 inches) to about 1.5 m (59 inches). In other implementations, the sensing device 200 may have a larger field of view, for example about ±65 degrees and may be sensitive to objects within a specified distance range from about 10-20 cm to 3 m.

The field of detection of the sensing device 200 (defined herein as the range of distances and angles within which the sensing device 200 is operable to detect vital signs of a patient with an acceptable level of performance) depends on the specific configuration of the sensing device (e.g., type of sensor, sensitivity, range, frequency of operation, signal processing techniques) and may in some cases be limited by interference or noise from other sources, such as other reflections from other sensing devices of nearby patients, as well as from objects and/or other electromagnetic sources. In addition, if the sensing device 200 is in a fixed location relative to the room, for example mounted on a wall of the room, the patient may or may not be within the field of detection of the sensing device 200 depending on the position of the bed 100 within the room. For example, if the patient moves within the bed, the relevant part of the patient (for example, the torso for heart rate and breathing) might no longer be within the field of detection of a sensing device 200 that was previously trained on the patient, thereby resulting in erroneous or missed readings. The anthropomorphic measurements (height, weight and body surface area) of the patient may also affect whether the patient's position in the bed is within the field of detection of the sensing device 200. In addition, the sensing device 200, like any other piece of equipment monitoring a patient in a hospital bed, should preferably be unobtrusive to hospital staff who need to interact with the patient, and regularly clean or maintain the bed.

The following embodiments provide for different configurations of systems, patient support apparatuses and sensing devices to alleviate some of the aforementioned challenges.

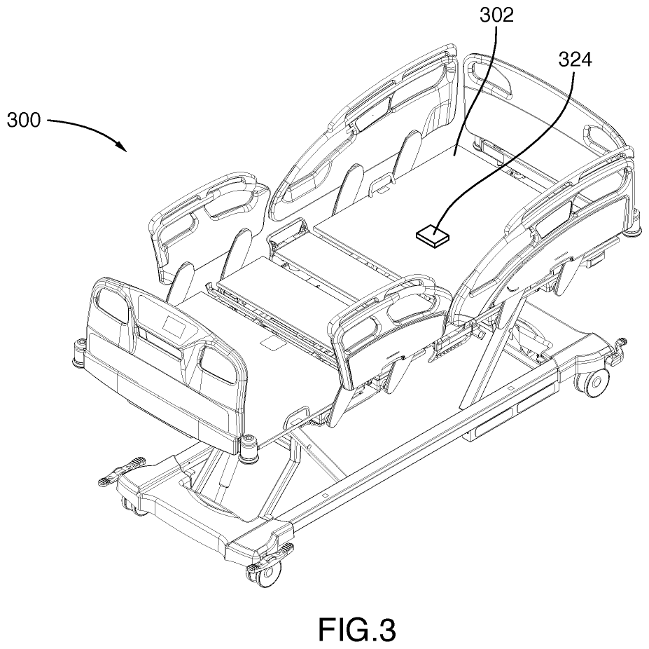

Referring now to FIG. 3, a hospital bed 300 will be described according to an embodiment. Most of the features of the hospital bed 300 are similar to those of the hospital bed 100, and will not be described again in detail.

The hospital bed 300 is equipped with a sensing device 324 that may be similar in operation to the sensing device 200 previously described. The sensing device 324 is operable to detect one or more vital signs of a patient when the patient is within the patient area of the bed 300. In this embodiment, the sensing device 324 is coupled to the backrest 302 of the bed 300, preferably disposed on the underside of the backrest 302 of the bed 300, and is configured to move with the backrest 302. If the sensing device 324 is disposed on the underside of the backrest 302, the backrest 302 is preferably made of a material that is substantially transparent or permeable to the electromagnetic wavelengths used by the sensing device 324. Such materials allow electromagnetic waves to pass through with minimal attenuation and/or reflection. It will be appreciated that factors that may affect transparency of the materials depend on inter alia the frequency of the electromagnetic waves, and material characteristics of the materials used (e.g., composition, thickness, etc.). As a non-limiting example, for UWB frequencies, the materials of the components in which the sensing device 324 is integrated may include plexiglass, plastics, wood, and the like.

In this arrangement, the patient is likely to remain within the effective range of the sensing device 324, because the sensing device 324 remains at a consistent vertical distance from the patient, being separated by approximately the thickness of the mattress and the backrest 102.

The sensing device 324 may optionally comprise one or more communication interface(s) operable to transmit vital sign readings to computing devices. The computing devices may be connected to the sensing device 324 via a communication network (not shown). The communication network may include a hospital network, for example connected to the sensing device 324 via a wired or wireless connection, either directly or via the hospital bed 300 if the hospital bed 300 has network connectivity. The patient may then be monitored remotely, for example from a computing device located at a nursing station.

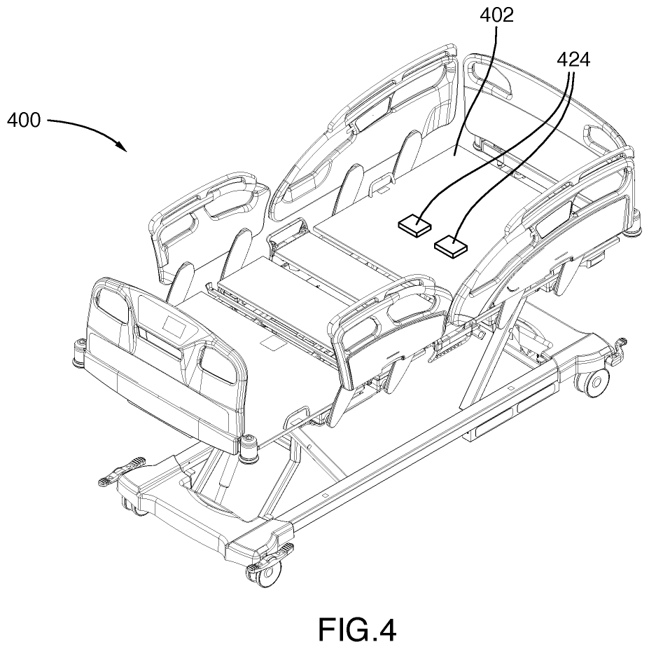

Referring now to FIG. 4, hospital bed 400 will be described according to an embodiment. Most of the features of the hospital bed 400 are similar to those of the hospital bed 100, and will not be described again in detail.

The hospital bed 400 is equipped with two or more spaced apart sensing devices 424, two of which are shown, coupled to the backrest 402 of the hospital bed 400 similarly to the embodiment of FIG. 3. Each sensing device 424 may be similar in operation to the sensing device 200 previously described. In this configuration, the sensing devices 424 have non-overlapping fields of view, thereby providing improved coverage of the patient area of the hospital bed 400. This embodiment may have an advantage compared to the embodiment of FIG. 3 in that the combination of multiple sensing devices 424 is able to cover a wider portion of the patient area, thereby being more likely to capture valid vital sign readings if the patient changes position within the patient area of the hospital bed 400.

The sensing devices 424 may optionally comprise one or more communication interface(s) operable to transmit vital sign readings to computing devices (not shown). The computing devices may be connected to the sensing device 324 via a communication network (not shown). The communication network may include a hospital network, and the sensing devices 424 may be connected for example via a wired or wireless connection, either directly or via the hospital bed 400 if the hospital bed 400 has network connectivity. The patient may then be monitored remotely, for example from a nursing station. Optional software, which may be stored and executed in the sensing devices 424, the hospital bed 400, or elsewhere in the network, may be used to select the sensing device 424 having the readings most likely to correspond to accurate human vital signs, and display only the readings from the selected sensing device 424 to medical personnel.

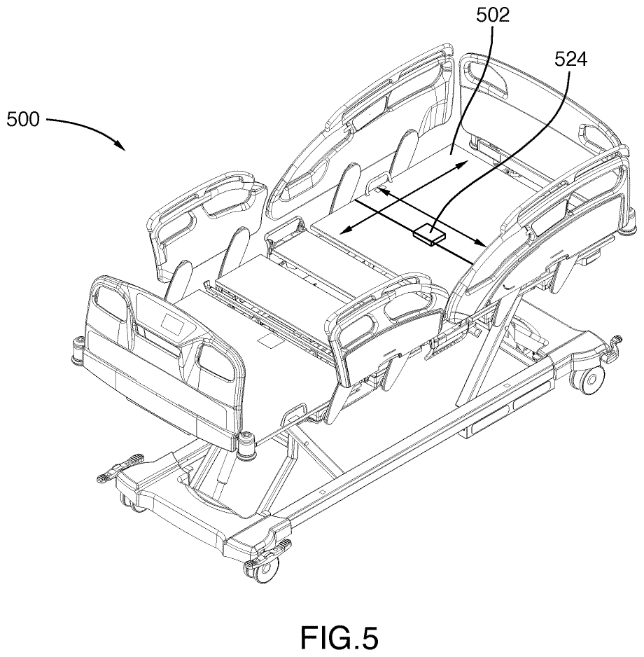

Referring now to FIG. 5, hospital bed 500 will be described according to an embodiment. Most of the features of the hospital bed 500 are similar to those of the hospital bed 100, and will not be described again in detail.

The hospital bed 500 is equipped with a sensing device 524 mounted on an adjustable positioning mechanism 520 coupled to the backrest 502 of the hospital bed 500. The sensing device 524 may be similar in operation to the sensing device 200 previously described. The adjustable positioning mechanism 520 is configured to position the sensing device 524 along one or two axes.

The adjustable positioning mechanism 520 may include one or more of a horizontal sliding mechanism, a vertical elevation mechanism, a pivoting arm, a rotating platform, a telescopic extension, a multi-axis gimbal, a swiveling mount, a track system, a spring-loaded adjustment, and a remote-controlled operation, each configured to move the sensor at different positions and/or angles towards a portion of the body patient in the hospital bed 500. The adjustable positioning mechanism 520 may include one or more motors and a processing unit (e.g., microcontroller) to control and adjust the position and/or the angle of the sensing device 524.

When the hospital bed 500 detects that there is a patient in the patient area, for example by way of one or more weight sensors, the sensing device 524 can be configured to move to a position in which a suitably good reading of the patient's vital signs can be detected. If the hospital bed 500 has multiple weight sensors, for example near each corner of the patient area, such that the hospital bed 500 can estimate the position of the patient within the patient area, it may be possible for the sensing device 524 to be moved to a position that is based on an estimated position of the patient, to improve the quality of the vital sign readings.

This embodiment may have an advantage compared to the embodiment of FIG. 4 in that a single sensing device 524 is able to cover a wider portion of the patient area, and its position can be adjusted to adapt to the position of the patient in the hospital bed 500, thereby being more likely to capture valid vital sign readings if the patient changes position within the patient area of the hospital bed 500.

The sensing device 524 may optionally comprise one or more communication interface(s) operable to transmit vital sign readings to computing devices (not shown). The computing devices may be connected to the sensing device 524 via a communication network (not shown). The communication network may include a hospital network, and the sensing devices 524 may be connected for example via a wired or wireless connection, either directly or via the hospital bed 500 if the hospital bed 500 has network connectivity. The patient may then be monitored remotely, for example from a nursing station.

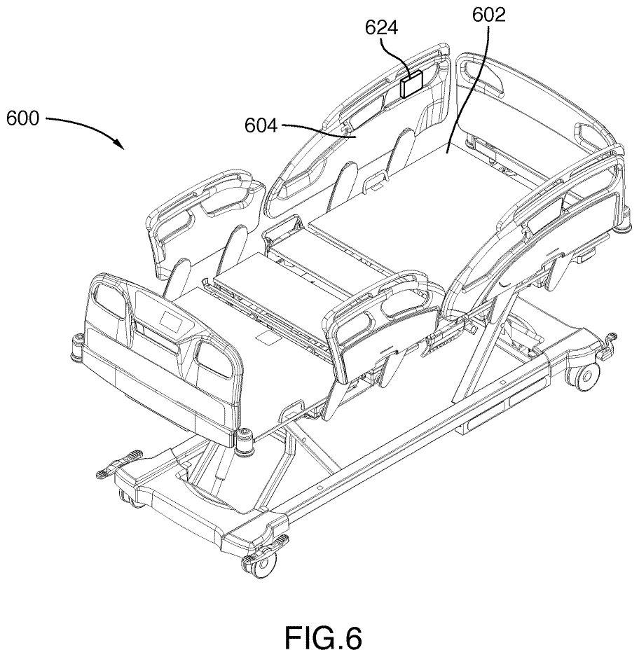

Referring now to FIG. 6, hospital bed 600 will be described according to an embodiment. Most of the features of the hospital bed 600 are similar to those of the hospital bed 100, and will not be described again in detail.

The hospital bed 600 is equipped with a sensing device 624 that may be similar in operation to the sensing device 200 previously described. The sensing device 624 is configured for detecting one or more vital signs of a patient when the patient is within the patient area of the bed 600. In this embodiment, the sensing device 624 is coupled to the siderail 604 of the bed 600, and is able to move with the siderail 604. If the sensing device 624 is installed inside the siderail 604, at least a portion of the siderail 604 is preferably made of a material that is substantially transparent to the wavelengths used by the sensing device 624 as explained above.

In this arrangement, the patient is likely to remain within the effective range of the sensing device 624, because the sensing device 624 remains at a consistent vertical distance from the patient, being configured to move up and down with the backrest 602.

The sensing device 624 may optionally comprise one or more communication interface(s) operable to transmit vital sign readings to computing devices (not shown). The computing devices may be connected to the sensing device 624 via a communication network (not shown). The communication network may include a hospital network, and the sensing devices 624 may be connected for example via a wired or wireless connection, either directly or via the hospital bed 600 if the hospital bed 600 has network connectivity. The patient may then be monitored remotely, for example from a nursing station.

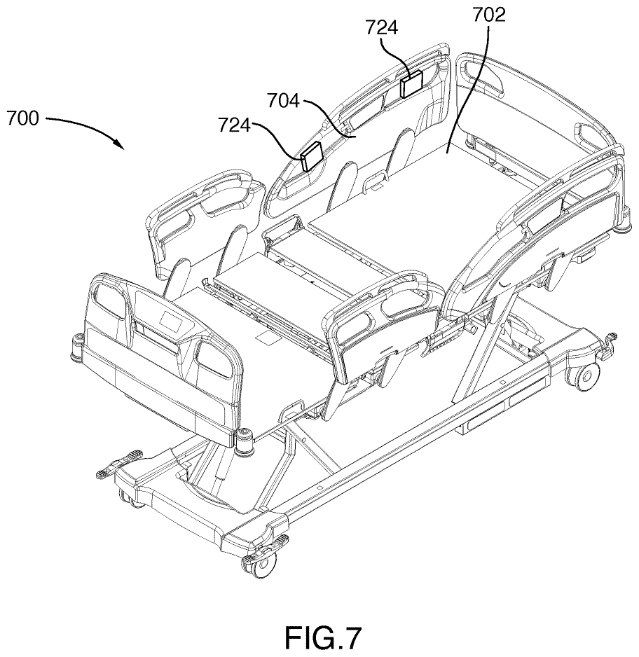

Referring now to FIG. 7, hospital bed 700 will be described according to an embodiment. Most of the features of the hospital bed 700 are similar to those of the hospital bed 100, and will not be described again in detail.

The hospital bed 700 is equipped with a plurality of sensing devices 724 (two of which are shown) that may be similar in operation to the sensing device 200 previously described. The sensing devices 724 are configured for detecting one or more vital signs of a patient when the patient is within the patient area of the bed 700. In this embodiment, the sensing devices 724 are coupled to the siderail 704 of the bed 700, and are able to move with the siderail 704, for example if the siderail 704 can be raised or lowered along with the backrest 702. If the sensing devices 724 are installed inside the siderail 704, at least a portion of the siderail 704 is preferably made of a material that is substantially transparent to the wavelengths used by the sensing devices 724, as explained above.

In this configuration, the sensing devices 724 have non-overlapping fields of view, thereby providing improved coverage of the patient area of the hospital bed 700. In this arrangement, the patient is likely to remain within the effective range of the sensing devices 724, because the sensing devices 724 remain at a consistent vertical distance from the patient, because the siderail 704 is configured to move up and down with the backrest 702.

This embodiment may have an advantage compared to the embodiment of FIG. 6 in that the combination of multiple sensing devices 724 is able to cover a wider portion of the patient area, thereby being more likely to capture valid vital sign readings if the patient changes position within the patient area of the hospital bed 700, as well as to adapt to patients of different height.

The sensing devices 724 may optionally comprise one or more communication interface(s) operable to transmit vital sign readings to computing devices (not shown). The computing devices may be connected to the sensing devices 724 via a communication network (not shown). The communication network may include a hospital network, and the sensing devices 724 may be connected for example via a wired or wireless connection, either directly or via the hospital bed 700 if the hospital bed 700 has network connectivity.

Optional software, which may be stored and executed in the sensing devices 724, the hospital bed 700, or elsewhere in the network, may be used to select the sensing device 724 having the readings most likely to correspond to accurate human vital signs, and display only the readings from the selected sensing device 724 to medical personnel.

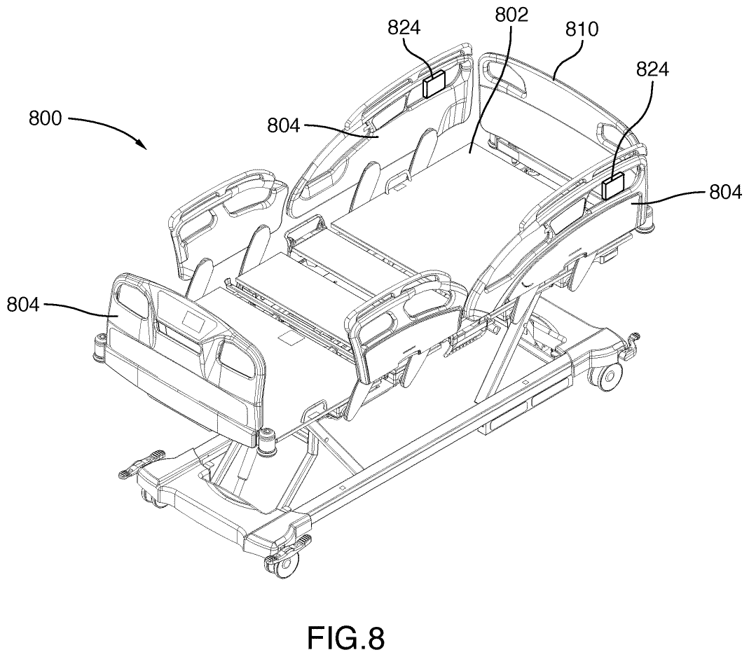

Referring now to FIG. 8, hospital bed 800 will be described according to an embodiment. Most of the features of the hospital bed 800 are similar to those of the hospital bed 100, and will not be described again in detail.

The hospital bed 800 is equipped with a plurality of sensing devices 824 (two of which are shown) that may be similar in operation to the sensing device 200 previously described. The sensing devices 824 are configured for detecting one or more vital signs of a patient when the patient is within the patient area of the bed 800. In this embodiment, at least one of the sensing devices 824 is coupled to each siderail 804 of the bed 800, and each sensing device 824 is able to move with the siderail 804 in which it is installed. If the sensing devices 824 are installed inside the siderails 804, at least a portion of each siderail 804 is preferably made of a material that is substantially transparent to the wavelengths used by the sensing devices 824, as explained above.

In this configuration, the sensing devices 824 have non-overlapping fields of view, thereby providing improved coverage of the patient area of the hospital bed 800. In this arrangement, the patient is likely to remain within the effective range of the sensing devices 824, because the sensing devices 824 remain at a consistent vertical distance from the patient, because the siderails 804 are configured to move up and down with the backrest 802. In addition, the sensing devices 824 would be less sensitive to the patient moving out of their effective distance range, because a patient moving away from one sensing device 824 would be moving toward the sensing device 824 on the opposite siderail 804. In addition, the presence of at least one sensing device 824 on each siderail 804 permits the capture of vital sign readings from one siderail 804 even if the other siderail 804 is lowered.

Optionally, the sensing devices 824 could be staggered along the length of the hospital bed 800, such that the sensing device 824 on one siderail 804 is closer to the headboard 810 and the sensing device 824 on the opposite siderail 804 is closer to the footboard 812. This may, in some examples provide improved coverage of the patient area of the hospital bed 800.

This embodiment may have an advantage compared to the embodiment of FIG. 6 in that the combination of multiple sensing devices 824 is able to cover a wider portion of the patient area, thereby being more likely to capture valid vital sign readings if the patient changes position within the patient area of the hospital bed 800. This embodiment may have an advantage compared to the embodiment of FIG. 7 in that the combination of multiple sensing devices 824 on both siderails 804 may be better able to capture valid vital sign readings if the patient is very close to one siderail 804.

The sensing devices 824 may optionally comprise one or more communication interface(s) operable to transmit vital sign readings to computing devices (not shown). The computing devices may be connected to the sensing devices 824 via a communication network (not shown). The communication network may include a hospital network, and the sensing devices 824 may be connected for example via a wired or wireless connection, either directly or via the hospital bed 800 if the hospital bed 800 has network connectivity. The patient may then be monitored remotely, for example from a nursing station. Optional software, which may be stored and executed in the sensing devices 824, the hospital bed 800, or elsewhere in the network, may be used to select the sensing device 824 having the readings most likely to correspond to accurate human vital signs, and display only the readings from the selected sensing device 824 to medical personnel.

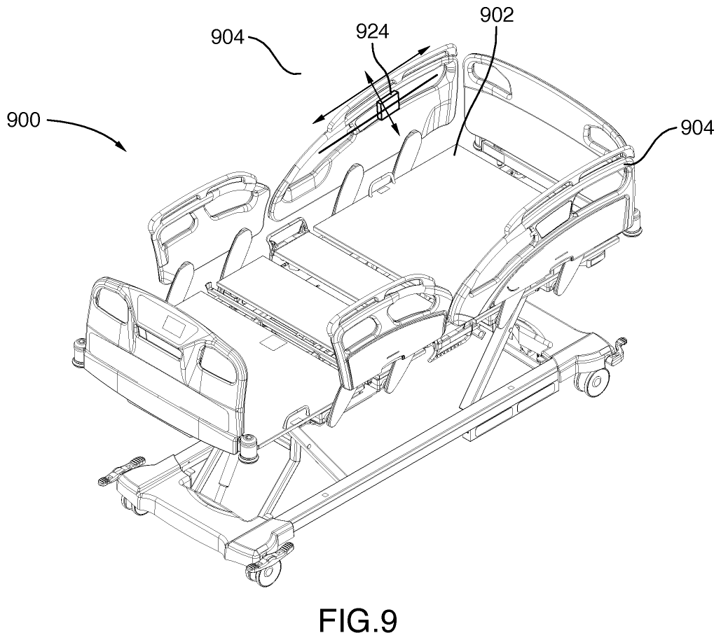

Referring now to FIG. 9, hospital bed 900 will be described according to an embodiment. Most of the features of the hospital bed 900 are similar to those of the hospital bed 100, and will not be described again in detail.

The hospital bed 900 is equipped with a sensing device 924 mounted on an adjustable positioning mechanism 920 coupled to the siderail 904 of the hospital bed 900. The sensing device 924 may be similar in operation to the sensing device 200 previously described.

The adjustable positioning mechanism 920 is configured to position the sensing device 924 along one or two axes. When the hospital bed 900 detects that there is a patient in the patient area, for example by way of one or more weight sensors within the bed 900 or other types of sensors (e.g., camera), the sensing device 924 can be configured to move to a position in which a suitably good reading of the patient's vital signs can be detected. If the hospital bed 900 has multiple weight sensors, for example near each corner of the patient area, such that the hospital bed 900 can estimate the position of the patient within the patient area, the adjustable positioning mechanism 920 may adjust the sensing device 924 to a position that is based on an estimated position of the patient, to improve the quality of the vital sign readings.

The adjustable positioning mechanism 920 may include one or more of a horizontal sliding mechanism, a vertical elevation mechanism, a pivoting arm, a rotating platform, a telescopic extension, a multi-axis gimbal, a swiveling mount, a track system, a spring-loaded adjustment, and a remote-controlled operation, each configured to move the sensor at different positions and/or angles towards a portion of the body patient in the hospital bed 900. The adjustable positioning mechanism 920 may include one or more motors and a processing unit (e.g., microcontroller) to control and adjust the position and/or the angle of the sensing device 924.

In this embodiment, the sensing device 924 is able to move with the siderail 904, for example if the siderail 904 can be raised or lowered along with the backrest 902. If the sensing device 924 is installed inside the siderail 904 (i.e., integrated within the siderail 904), at least a portion of the siderail 904 is preferably made of a material that is substantially transparent to the wavelengths used by the sensing devices 924, as explained above.

It will be appreciated that that in this embodiment, a single sensing device 924 with the adjustable positioning mechanism 920 is able to cover a wider portion of the patient area, and its position can be adjusted to adapt to the position of the patient in the hospital bed 500, thereby being more likely to capture valid vital sign readings if the patient changes position within the patient area of the hospital bed 900. It is contemplated that additional coverage may be achieved in some embodiments by providing a second sensing device 924 in the opposite siderail 904 that is also movable along one or two axes in a similar way.

The sensing device 924 may optionally comprise one or more communication interface(s) operable to transmit vital sign readings to computing devices (not shown). The computing devices may be connected to the sensing device 924 via a communication network (not shown). The communication network may include a hospital network, and the sensing devices 924 may be connected for example via a wired or wireless connection, either directly or via the hospital bed 900 if the hospital bed 900 has network connectivity.

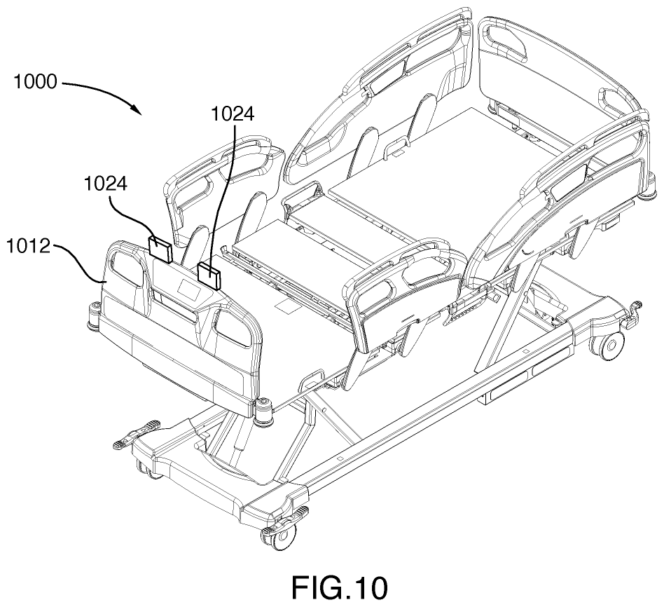

Referring now to FIG. 10, hospital bed 1000 will be described according to an embodiment. Most of the features of the hospital bed 1000 are similar to those of the hospital bed 100, and will not be described again in detail.

The hospital bed 1000 is equipped with one or more sensing devices 1024, two of which are shown, coupled to the footboard 1012 of the hospital bed 1000. If two or more sensing devices 1024 are provided, they may be spaced apart along the footboard 1012. In this configuration, the sensing devices 1024 have non-overlapping fields of view, thereby providing improved coverage of the patient area of the hospital bed 1000. Each sensing device 1024 may be similar in operation to the sensing device 200 previously described. This embodiment may have an advantage compared to other embodiments in that the footboard 1012 is sometimes a modular and easily interchangeable component of the hospital bed 1000, thereby making it convenient to retrofit the sensing devices 1024 into existing hospital bed 1000, or upgrade or service sensing devices 1024 already provided in a hospital bed 1000. It will be appreciated that in this embodiment, a patient's torso is more likely to be in the field of detection of the sensing devices 1024 in a typical lying or sleeping position, thereby ensuring that the patient's torso is within the effective distance range of the sensing devices 1024.

The sensing devices 1024 may optionally comprise one or more communication interface(s) operable to transmit vital sign readings to computing devices (not shown). The computing devices may be connected to the sensing devices 1024 via a communication network (not shown). The communication network may include a hospital network, and the sensing devices 1024 may be connected for example via a wired or wireless connection, either directly or via the hospital bed 1000 if the hospital bed 1000 has network connectivity. The patient may then be monitored remotely, for example from a nursing station. Optional software, which may be stored and executed in the sensing devices 1024, the hospital bed 1000, or elsewhere in the network, may be used to select the sensing device 1024 having the readings most likely to correspond to accurate human vital signs, and display only the readings from the selected sensing device 1024 to medical personnel.

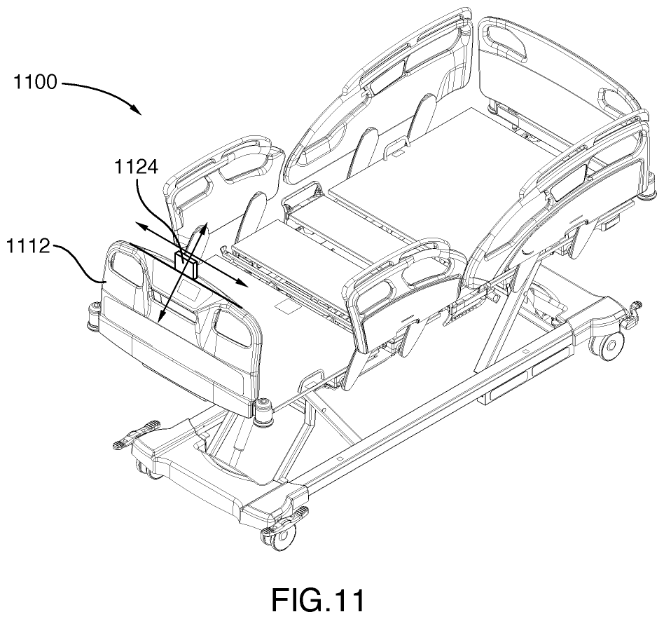

Referring now to FIG. 11, hospital bed 1100 will be described according to an embodiment. Most of the features of the hospital bed 1100 are similar to those of the hospital bed 100, and will not be described again in detail.

The hospital bed 1100 is equipped with a sensing device 1124, disposed in the footboard 1112 of the hospital bed 1100. The sensing device 1124 is configured for translational movement along one or two axes, for example by way of one or more motors (not shown) which may be coupled to the sensing device 1124 in any suitable manner. The sensing device 1124 may be similar in operation to the sensing device 200 previously described. When the hospital bed 1100 detects that there is a patient in the patient area, for example by way of one or more weight sensors, an adjustable mechanism (not shown), similar to the previously described adjustable mechanisms, can move the sensing device 1124 a position in which a suitably good reading of the patient's vital signs can be detected. If the hospital bed 1100 has multiple weight sensors, for example near each corner of the patient area, such that the hospital bed 1100 can estimate the position of the patient within the patient area, it may be possible for the sensing device 1124 to be moved to a position that is based on an estimated position of the patient, to improve the quality of the vital sign readings.

This embodiment may have an advantage compared to the embodiment of FIG. 10 in that a single sensing device 1124 is able to cover a wider portion of the patient area, and its position can be adjusted to improve the quality of readings, thereby being more likely to capture valid vital sign readings if the patient changes position within the patient area of the hospital bed 1100.

The sensing device 1124 may optionally comprise one or more communication interface(s) operable to transmit vital sign readings to computing devices (not shown). The computing devices may be connected to the sensing device 1124 via a communication network (not shown). The communication network may include a hospital network, and the sensing devices 1124 may be connected for example via a wired or wireless connection, either directly or via the hospital bed 1100 if the hospital bed 1100 has network connectivity. The patient may then be monitored remotely, for example from a nursing station.

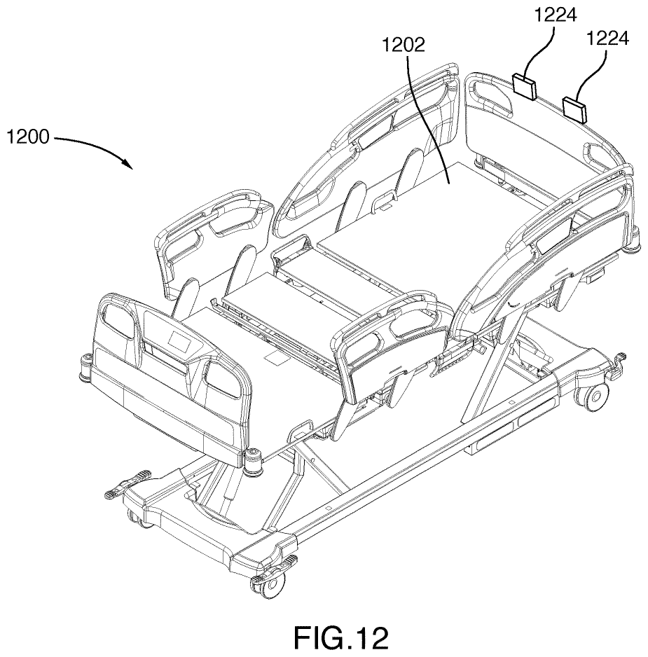

Referring now to FIG. 12, hospital bed 1200 will be described according to an embodiment. Most of the features of the hospital bed 1200 are similar to those of the hospital bed 100, and will not be described again in detail.

The hospital bed 1200 is equipped with one or more sensing devices 1224, two of which are shown, coupled to a support structure mounted to the backrest 1202 of the hospital bed 1200. If two or more sensing devices 1224 are provided, they may be spaced apart along the backrest. In this configuration, the sensing devices 1224 have non-overlapping fields of view, thereby providing improved coverage of the patient area of the hospital bed 1200. Each sensing device 1224 may be similar in operation to the sensing device 200 previously described. This embodiment may have an advantage compared to the embodiments of FIGS. 6-9 that the patient's torso is more likely to be at a consistent distance from the sensing devices 1224 in a typical lying or sleeping position, thereby ensuring that the patient's torso is within the effective distance range of the sensing devices 1224.

The sensing device 1224 may optionally comprise one or more communication interface(s) operable to transmit vital sign readings to computing devices (not shown). The computing devices may be connected to the sensing device 1224 via a communication network (not shown). The communication network may include a hospital network, and the sensing devices 1224 may be connected for example via a wired or wireless connection, either directly or via the hospital bed 1200 if the hospital bed 1200 has network connectivity. The patient may then be monitored remotely, for example from a nursing station. Optional software, which may be stored and executed in the sensing devices 1224, the hospital bed 1200, or elsewhere in the network, may be used to select the sensing device 1224 having the readings most likely to correspond to accurate human vital signs, and display only the readings from the selected sensing device 1224 to medical personnel.

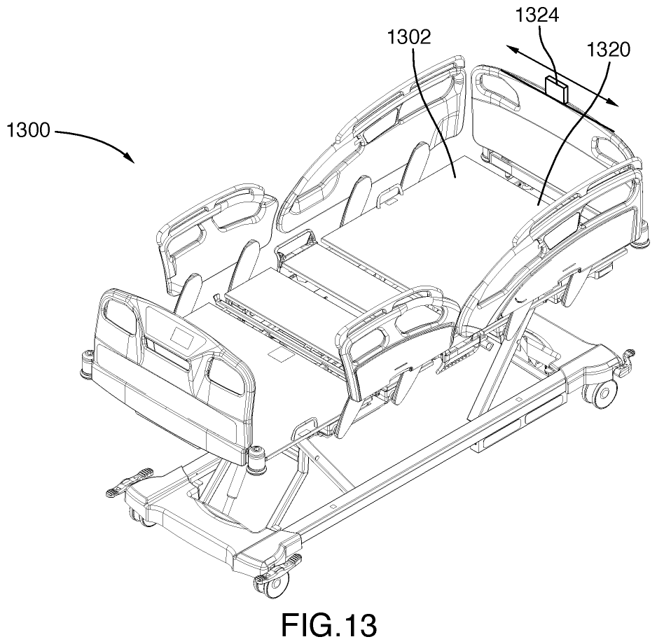

Referring now to FIG. 13, hospital bed 1300 will be described according to an embodiment. Most of the features of the hospital bed 1300 are similar to those of the hospital bed 100, and will not be described again in detail.

The hospital bed 1300 is equipped with a sensing device 1324, disposed on a support structure mounted to an adjustable positioning mechanism 1320 coupled to the backrest 1302 of the hospital bed 1300. The adjustable positioning mechanism 1320 is configured to position the sensing device 1324 along one or two axes. The sensing device 1324 may be similar in operation to the sensing device 200 previously described.

The adjustable positioning mechanism 1320 may include one or more of a horizontal sliding mechanism, a vertical elevation mechanism, a pivoting arm, a rotating platform, a telescopic extension, a multi-axis gimbal, a swiveling mount, a track system, a spring-loaded adjustment, and a remote-controlled operation, each configured to move the sensor at different positions and/or angles towards a portion of the body patient in the hospital bed 1300. The adjustable positioning mechanism 1320 may include one or more motors and a processing unit (e.g., microcontroller) to control and adjust the position and/or the angle of the sensing device 1324.

It will be appreciated that in this embodiment, a single sensing device 1324 provided with an adjustable positioning mechanism 1320 is able to cover a wider portion of the patient area, and its position can be adjusted to improve the quality of readings, thereby being more likely to capture accurate vital sign readings if the patient changes position within the patient area of the hospital bed 1300.

The sensing device 1324 may optionally comprise one or more communication interface(s) operable to transmit vital sign readings to computing devices (not shown). The computing devices may be connected to the sensing device 1324 via a communication network (not shown). The communication network may include a hospital network, and the sensing devices 1324 may be connected for example via a wired or wireless connection, either directly or via the hospital bed 1300 if the hospital bed 1300 has network connectivity. The patient may then be monitored remotely, for example from a nursing station.

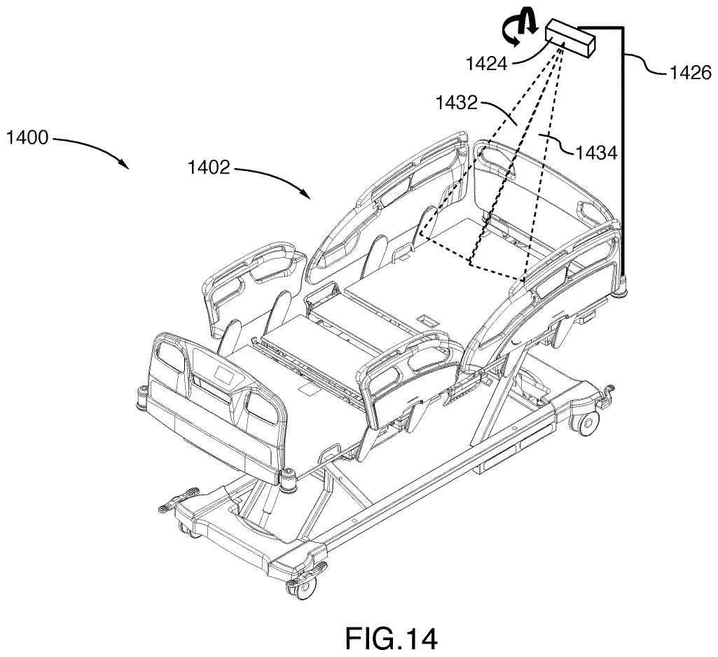

Referring now to FIG. 14, system 1400 comprising hospital bed 1402 and sensing device 1424 will be described according to an embodiment. Most of the features of the hospital bed 1402 are similar to those of the hospital bed 100, and will not be described again in detail.

A sensing device 1424 is mounted above the hospital bed 1402 via a stand 1426 that may be the same type of stand typically used as a bed-mounted IV pole, i.e., a vertical support configured for supporting an object above the patient platform of the hospital bed 1402.

The sensing device 1424 may be pivotable using an angular adjustment mechanism (not shown) to be aimed at the patient, i.e., to include the patient within the field of detection of the sensing device 1424. The sensing device 1424 may be manually aimed at the patient, for example in the case of an unconscious or otherwise immobile patient. The sensing device 1424 may alternatively be pivotable using an automated angular adjustment mechanism controlled by actuators (not shown) which may be coupled to the sensing device 1424 in any suitable manner. The angular adjustment mechanism may comprise a pivoting joint, a swivel mount, or a multi-axis gimbal system, each configured to enable rotation of the sensing device about one or more axes to cover the patient area. For example, the device can be rotated horizontally (azimuthal angle) to align with the width of the bed, or vertically (elevation angle) to align with the height of the patient. The angular adjustment mechanism may also include locking features to secure the sensing device at a desired angle to ensure stable and accurate measurements.

When the hospital bed 1402 detects that there is a patient in the patient area, for example by way of one or more weight sensors, the sensing device 1424 can be configured to pivot to an angle at which a suitably good reading of the patient's vital signs can be detected. If the hospital bed 1402 has multiple weight sensors, for example near each corner of the patient area, such that the hospital bed 1402 can estimate the position of the patient within the patient area, it may be possible for the sensing device 1424 to be pivoted to an angle that is based on an estimated position of the patient, to improve the quality of the vital sign readings.

Optionally, the stand 1426 could support two or more sensing devices 1424, for example in a single housing, that are oriented at slightly different angles so as to have non-overlapping fields of detection 1432, 1434, to achieve a better angular field of view and limit or remove the need to pivot the sensing device 1424.

The sensing device 1424 may optionally transmit vital sign readings to other computing devices, to a hospital network, for example via a wired or wireless connection, either directly or via the hospital bed 1402 if the hospital bed 1402 has network connectivity. The patient may then be monitored remotely, for example from a nursing station.

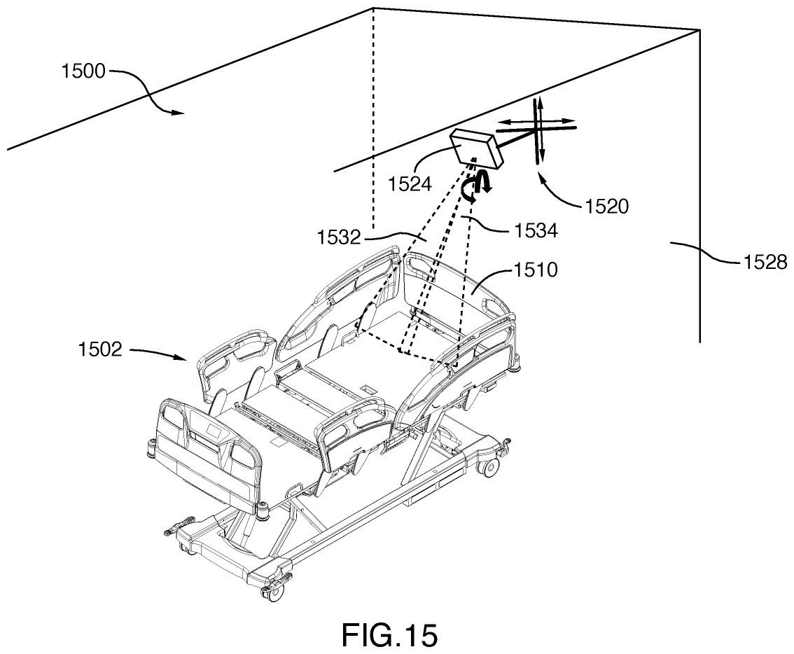

Referring now to FIG. 15, a system 1500 for positioning a sensing device 1524 relative to a patient in hospital bed 1502 by using adjustable positioning mechanism 1520 will be described according to an embodiment. Most of the features of the hospital bed 1502 are similar to those of the hospital bed 100, and will not be described again in detail.

A sensing device 1524 is mounted to an adjustable positioning mechanism 1520. The adjustable positioning mechanism 1520 is mounted above the hospital bed 1502, to the wall 1528 that would be behind the headboard 1510 when the hospital bed 1502 is in the typical position in the room. Alternatively, the adjustable positioning mechanism 1520 may be mounted to a different wall in the room.

The adjustable positioning mechanism 1520 is configured to pivot the sensing device 1524 to be aimed at the patient, i.e., to include the patient within the field of detection of the sensing device 1524. The adjustable positioning mechanism 1520 may also be configured to perform translational movement of the sensing device 1524 along the wall 1528 in one or more directions.

The adjustable positioning mechanism 1520 may include one or more of a horizontal sliding mechanism, a vertical elevation mechanism, a pivoting arm, a rotating platform, a telescopic extension, a multi-axis gimbal, a swiveling mount, a track system, a spring-loaded adjustment, and a remote-controlled operation, each configured to move the sensor at different positions and/or angles towards a portion of the body patient in the hospital bed 1502. The adjustable positioning mechanism 1520 may include one or more motors and a processing unit (e.g., microcontroller) to control and adjust the position and/or the angle of the sensing device 1524.

When the hospital bed 1502 or another sensor associated thereto detects that there is a patient in the patient area, for example by way of one or more weight sensors, the adjustable positioning mechanism 1520 can be configured to move the sensing device 1524 to a position and an angle at which a suitably good reading of the patient's vital signs can be detected. If the hospital bed 1502 has multiple weight sensors, for example near each corner of the patient area, such that a processing unit associated with the hospital bed 1502 can estimate the position of the patient within the patient area, it may be possible for the sensing device 1524 to be moved to a position and an angle that is based on an estimated position of the patient, to improve the quality of the vital sign readings.

Optionally, the wall 1528 could support two or more sensing devices 1524, for example in a single housing, that are oriented at slightly different angles so as to have non-overlapping fields of detection 1532, 1534, to achieve a better angular field of view and limit or remove the need to pivot the sensing device 1524.

The sensing device 1524 may optionally transmit vital sign readings to other computing devices (not shown), to a hospital network, for example via a wired or wireless connection, either directly or via the hospital bed 1502 if the hospital bed 1502 has network connectivity. The patient may then be monitored remotely, for example from a nursing station.

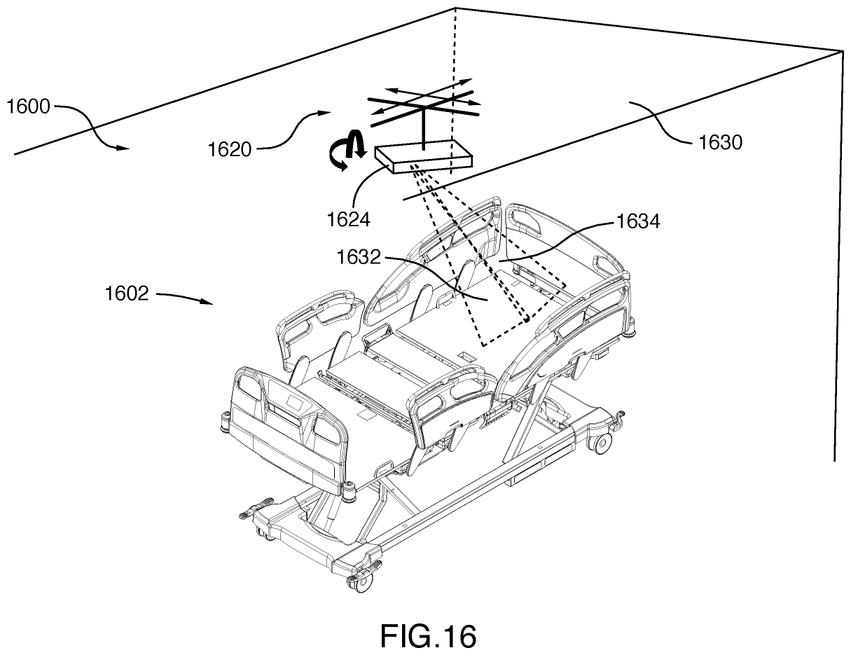

Referring now to FIG. 16, a system 1600 for positioning a sensing device 1624 relative to a hospital bed 1602 by using an adjustable positioning mechanism 1620 will be described according to an embodiment. Most of the features of the hospital bed 1602 are similar to those of the hospital bed 100, and will not be described again in detail.

A sensing device 1624 is mounted to an adjustable positioning mechanism 1620. The adjustable positioning mechanism 1620 is mounted above the hospital bed 1602, to the ceiling 1630 that would be above the hospital bed 1602 when the hospital bed 1602 is in the typical position in the room (e.g., zone within a room).

The adjustable positioning mechanism 1620 may include one or more of a horizontal sliding mechanism, a vertical elevation mechanism, a pivoting arm, a rotating platform, a telescopic extension, a multi-axis gimbal, a swiveling mount, a track system, a spring-loaded adjustment, and a remote-controlled operation, each configured to move the sensor at different positions and/or angles towards a portion of the body patient in the hospital bed 1602. The adjustable positioning mechanism 1620 may include one or more motors and a processing unit (e.g., microcontroller) to control and adjust the position and/or the angle of the sensing device 1624.

The adjustable positioning mechanism 1620 is configured to pivot the sensing device 1624 so that it can be aimed at the patient, i.e., to include the patient within the field of detection of the sensing device 1624. The adjustable positioning mechanism 1620 may also be configured to perform translational movement of the sensing device 1624 along the ceiling 1630 in one or more directions.

When the hospital bed 1602 or another sensor associated thereto detects that there is a patient in the patient area, for example by way of one or more weight sensors, the adjustable positioning mechanism 1620 can be configured to move the sensing device 1624 to a position and an angle at which a suitably good reading of the patient's vital signs can be detected. If the hospital bed 1602 has multiple weight sensors, for example near each corner of the patient area, such that a processing unit associated with the hospital bed 1602 can estimate the position of the patient within the patient area, it may be possible for the sensing device 1624 to be moved to a position and an angle that is based on an estimated position of the patient, to improve the quality of the vital sign readings.

Optionally, the ceiling 1630 could support two or more sensing devices 1624, for example in a single housing, that are oriented at slightly different angles so as to have non-overlapping fields of detection 1632, 1634, to achieve a better angular field of view and limit or remove the need to pivot the sensing device 1624.

The sensing device 1624 may optionally transmit vital sign readings to other computing devices (not shown), to a hospital network, for example via a wired or wireless connection, either directly or via the hospital bed 1602 if the hospital bed 1602 has network connectivity. The patient may then be monitored remotely, for example from a nursing station.

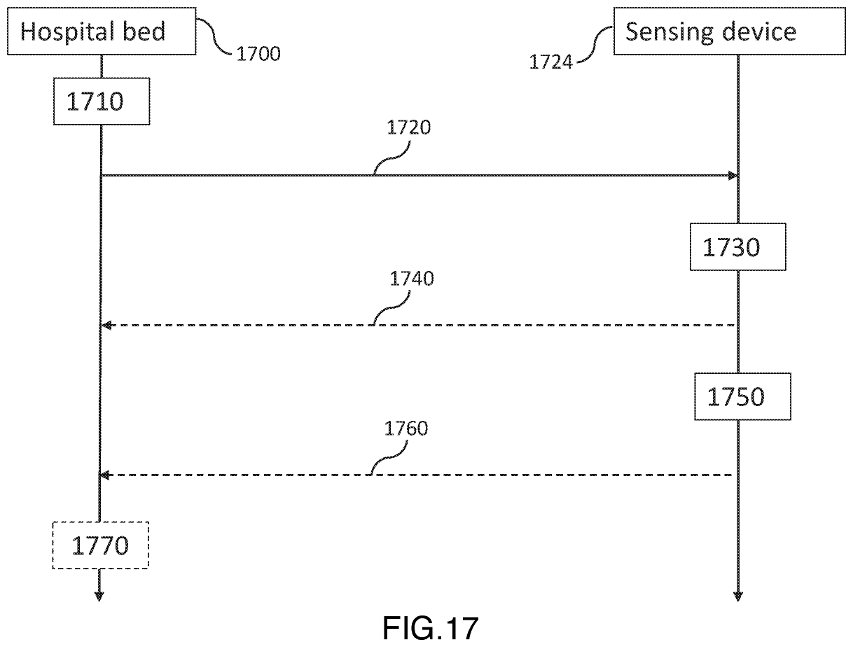

Referring now to FIG. 17, a method 1705 of orienting a sensing device will be described.

The method 1705 of orienting the sensing device may be executed within a system comprising the sensing device when the sensing device is mounted to an adjustable positioning mechanism configured to adjust the position and/or angle of the sensing device, such as the sensing devices 524, 924, 1124, 1324, 1424, 1524, and 1624. The adjustable positioning mechanism may include one or more motors and a processing unit (e.g., microcontroller) to control and adjust the position and/or the angle of the sensing device.

The method 1705 may be executed by a processing unit, such as the processing unit (e.g., controller) of the hospital bed 1700 and/or a processing unit of the adjustable positioning mechanism (not shown).

At step 1710, the processing unit associated with a hospital bed 1700 determines position information about a position of a patient within a patient area of the hospital bed. The hospital bed 1700 may be any of the hospital beds previously described. The position information may be an exact or approximate position of the patient within the patient area of the hospital bed 1700. The position information may be partial information about the position of the patient, for example the patient's position in the length direction of the hospital bed 1700 only, or the width direction only. The position information may be information about whether the patient is currently in the patient area of the hospital bed 1700. The position information may be information about whether a person in the patient area of the hospital bed 1700 is the patient designated to occupy the hospital bed 1700, for example based on biometric identification. The position information may be determined by any suitable means, such as weight sensors incorporated into different parts of the hospital bed 1700, for example integrated into the wheels or their supports. The position information may be determined by a pressure sensitive material disposed beneath the patient area, such as one or more pressure-sensitive mats sold under the Velostat™ name. In one or more alternative embodiments, other types of sensors such as cameras including optical and infrared cameras may be used to determine the position of the patient in the hospital bed 1700.

At step 1720, a signal indicative of the position of the patient is transmitted to an adjustable positioning mechanism to which the sensing device 1724 is mounted. The signal is received by a processing unit associated with the adjustable positioning mechanism (e.g., microcontroller of the bed or of the adjustable positioning mechanism). The signal transmitted at step 1720 does not necessarily include all of the position information determined at step 1710, and may include only partial information. For example, if the sensing device 1724 is only configured for one degree of movement, such as rotation about only one axis or translation in only one dimension, the signal may optionally be representative of only the position of the patient in the direction in which the sensing device 1724 is configured for movement. The sensing device 1724 may be integrated into the hospital bed 1700, for example as described with respect to FIG. 5, 9, 11, or 13. The sensing device 1724 may be a separate module coupled to the hospital bed 1700, for example as described with respect to FIG. 14. The sensing device 1724 may be mounted to a structure external to the hospital bed, for example as described with respect to FIG. 15 or 16.

At step 1730, the sensing device 1724 is oriented based on the received signal. The sensing device may orient itself by translation or by rotation, or both, depending on the movement capabilities of the sensing device 1724. The sensing device 1724 may be configured to select the movement or the final orientation such that the position indicated by the signal received at step 1720 is within the field of detection of at least one sensor of the sensing device 1724.

At step 1740, the sensing device 1724 optionally transmits a signal to the hospital bed 1700 confirming that the signal was received at step 1720, which may be a signal indicating that the sensing device 1724 has been successfully oriented based on the signal received at step 1720.

At step 1750, the sensing device 1724 determines information indicative of at least one vital sign of the patient. The at least one vital sign may include a heart rate (HR) and respiratory rate (RR) of the patient.

At step 1760, the sensing device 1724 optionally transmits to the hospital bed 1700 a signal containing the information indicative of the at least one vital sign of the patient.

At step 1770, the hospital bed 1700 optionally transmits to a communication network of the hospital the information indicative of the at least one vital sign of the patient. The information may then be monitored remotely by medical staff.

It is contemplated that these embodiments can be combined in any permutation. For example, a hospital bed could have sensing devices in one or both siderails of a bed and one or more of the headboard and footboard of the bed. In general, it may be advantageous to have sensing devices on two different sides of the hospital bed, for example on opposite sides of the hospital bed, to increase the combined field of detection of the sensing devices.

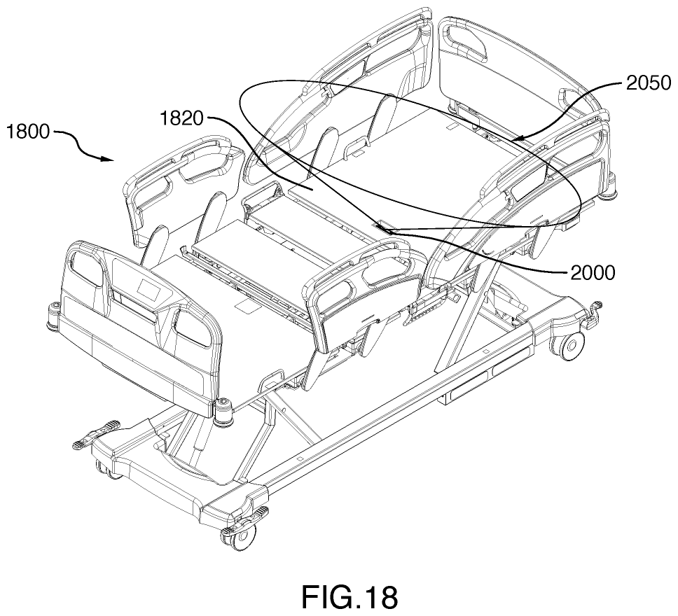

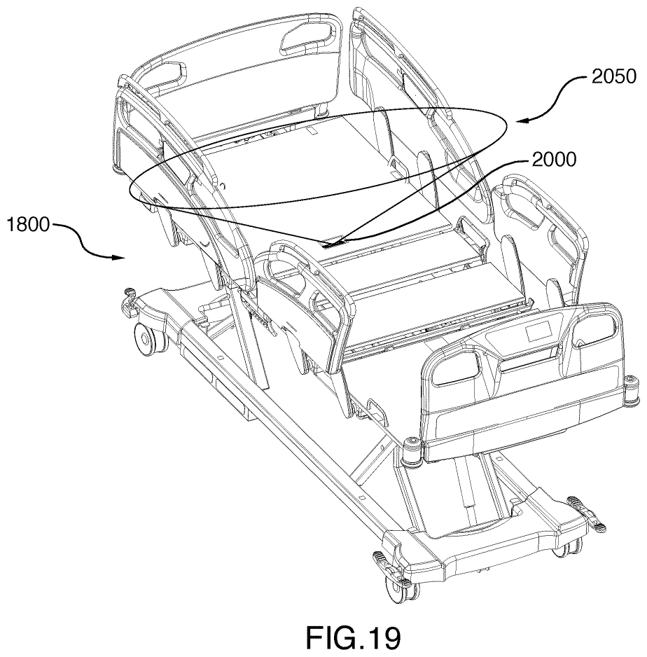

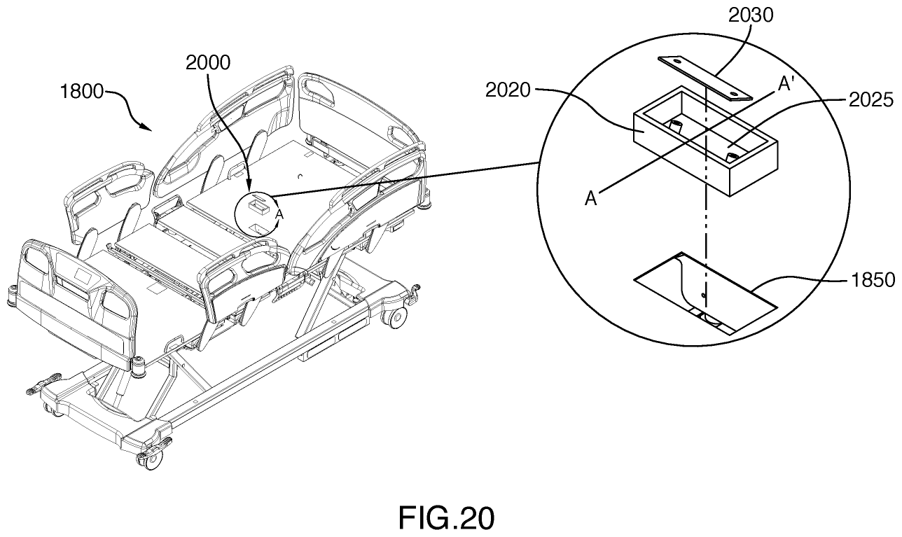

With reference to FIGS. 18 to 21, a hospital bed 1800 associated with sensing device 2000 will be described according to a fifteenth embodiment. Most of the features of the hospital bed 1800 are similar to those of the hospital bed 100 and will not be described again in detail. It will be appreciated that hospital bed 1800 is an embodiment of a patient support apparatus.

The hospital bed 1800 is equipped with the sensing device 2000 mounted to the backrest 1820 of the hospital bed 1800. The sensing device 2000 comprises a housing 2020, a support structure 2025 disposed within the housing 2020 and a sensor 2030 mounted on the support structure 2025.

In some embodiments, the housing 2020 of the sensing device 2000 may be coupled to the underside (i.e., bottom surface) of the backrest 1820 of the hospital bed 1800. The housing 2020 of the sensing device 2000 may be secured to the backrest 1820 of the hospital bed 1800 via one or more of a bracket, a mounting system, fastening elements, adhesive means and the like to ensure stability and durability even during the dynamic movements of the backrest 1820. In one or more alternative implementations, the housing 2020 of the sensing device may be fixed to another panel and/or location on or adjacent to the hospital bed 1800.

In the embodiment illustrated FIG. 18, the sensor 2030 is oriented at an angle within the housing 2020 using the support structure 2025 in the form of a bracket, and the housing 2020 is fixed to the backrest 1820 using fasteners in the form of screws (not shown). It will be appreciated that other alternatives may be possible.

In other embodiments, the housing 2020 of the sensing device 2000 may be secured to the backrest 1820 via an adjustable bracket and may be configured to move at an angle and optionally translational movement along one or two axes, for example by way of one or more motors (not shown) which may be coupled to the sensing device 2000 in any suitable manner.

The sensor 2030 is mounted at an angle on the support structure 2025 within the housing 2020. The angle may be chosen to optimize the detection and monitoring of vital signs of the patient in the hospital bed 1800, as will be explained below.

The housing 2020 is formed of a material that enables propagation of electromagnetic waves therethrough by minimizing attenuation and reflection of electromagnetic waves, particularly in the frequencies used by the sensor 2030. Non-limiting examples of such materials may include wood, plexiglass, plastic depending on the electromagnetic frequencies used by the sensor 2030. While the housing 2020 is depicted as being a rectangular box, it will be appreciated that the housing 2020 may have different shapes such as square, oval, and the like without departing from the scope of the present technology.

In the illustrated embodiment, the sensing device 2000 is integrated flush with a surface of the backrest via an opening 1850 shaped and dimensioned to receive the housing 2020 of the sensing device 2000.

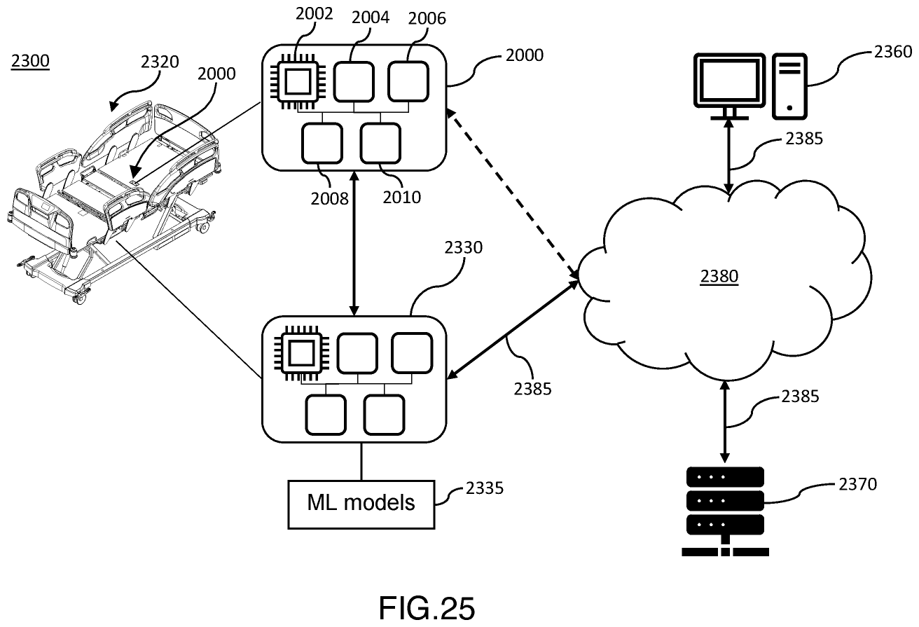

As best seen in FIG. 25, the sensor 2030 of the sensing device 2000 comprises components similar to the sensing device 200, i.e., one or more processing units 2002, memories 2004, transceivers 2006, input/output ports 2008 and communication interfaces 2010. The sensing device 2000 may be connected to the circuit of the hospital bed 1800 to receive electrical power therefrom (not shown). In one or more implementations, the sensing device 2000 may be powered by an energy source such as a battery.

In this embodiment, the sensing device 2000 is configured to transmit and receive UWB radar signals. As a non-limiting example, the sensing device 2000 may be configured to use 6.5 to 9 GHz impulse radio UWB radar with a radio signal emission angle of ±65 degrees (130 degrees total), which defines a field of view 2050. The field of view 2050 in combination with the detection range of the sensor 2030 (e.g., about 10 m) refers to the spatial region within which the sensor is configured to detect vital signs of the patient supported by the patient support apparatus, e.g., the hospital bed 1800. In the context of UWB technology, the field of detection is determined by the angular coverage and range of the UWB signals emitted and received by the sensor 2030 that enable detection of the one or more vital signs of the patient.

In the embodiment illustrated in FIGS. 18 to 21, the sensing device 2000 provides measurements of vital signs with a single sensor without the need to integrate a mechanism to automatically track the patient in bed.

In this example, the position and orientation of the sensing device 2000 enables targeting a specific region on the body of a patient supported by the hospital bed 1800. Developer(s) of the present technology have determined the specific target region following several measurements, which corresponds to the area of the lower back 2090 of the patient 2080, as shown in FIG. 22.

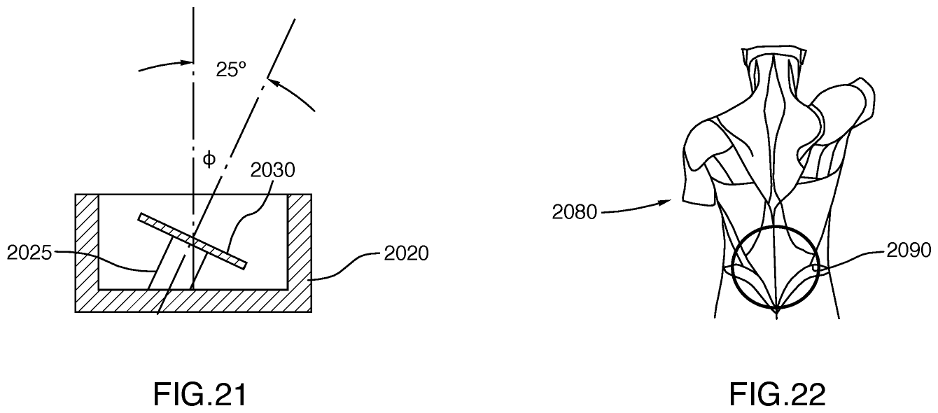

In the embodiment illustrated in FIGS. 18 to 21, the sensing device 2000 is centered between the left and right ends of the backplate and positioned at about 695 mm from the head end of the bed 1800. The sensor 2030 is oriented towards the upper body of the patient at an angle φ of 25 degrees within its housing 2020 relative to the backrest 1820 of the hospital bed 1800, as best seen in FIG. 21. The angle φ of 25 degrees has been determined by subtracting the emission angle of the sensor 2030 65 from 90.

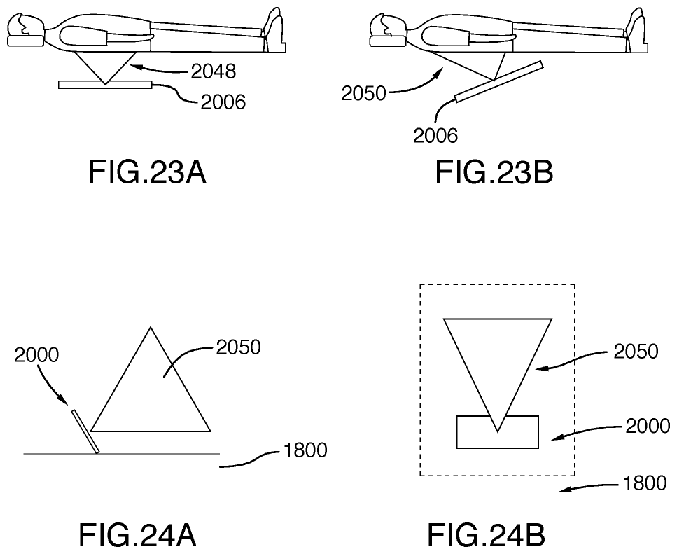

The angle of 25 degrees between the backrest and the sensor 2030 maximizes the field of detection, thereby improving detection of the vital signals of the patient irrespective of the anthropomorphic measurements of the patient, the position or posture of the patient within the hospital bed 1800, the configuration of the hospital bed 1800 and the location of the hospital bed 1800. The angle complements the opening angle of ±65 degrees of the sensor 2030, resulting in the lower part of the field of detection 2050 being parallel to the hospital bed 1800, irrespective of the configuration of the bed 1800, even when the head end of the bed is elevated to a near vertical or vertical position (e.g., high Fowler's position) as schematically shown in FIG. 24A. This ensures that the entire field of detection 2050 is directed towards the patient's body, and increases the field of detection 2050 of the sensing device 2000 compared to the configuration of FIG. 23A with field of detection 2048. It will be appreciated that this configuration enables detecting vital signs of the patient more accurately by including the upper body of the patient within the field of detection of the sensing device and by minimizing interference with the frame of the bed 1800.

In one or more implementations, the angle at which the sensor 2030 is oriented is determined by calculating (90−FOV_s), where FOV_s is the field of view of the sensor 2030.

It will be appreciated that while in this embodiment, the sensor 2030 is oriented within the housing 2020 of the sensing device 2000 at an angle using support structure 2025 (e.g., bracket), should the angle of the sensor 2030 correspond to the angle of the housing 2020 of the sensing device 2000, the housing 2020 may be coupled to the bed 1800 at the selected angle.

Further, it is assumed that the angle reduces interference caused by the metal backplate at the base of the bed, as shown in FIG. 24A and FIG. 24B.

It will be appreciated that by minimizing interference and by using the configuration described with reference to FIGS. 18 to 24, the vital signs of patients may be detected by the sensing device 2000 regardless of the patient anthropomorphic measurements (weight, height, and patient body surface area), position, and mattress type (e.g., air or foam mattress).

With reference to FIG. 25, an environment and system 2300 comprising a hospital bed 2320 equipped with a sensing device 2000, a nurse station computer 2360, a server 2370, coupled to a communication network 2380 via respective communication links 2385.

Most of the features of the hospital bed 2320 are similar to those of the hospital bed 100 and will not be described again in detail. It will be appreciated that hospital bed 2320 is an embodiment of a patient support apparatus.

The hospital bed 2320 may be located in a healthcare facility. The hospital bed 2320 may be for example located within a zone of a room, a hallway, an intensive care unit, an emergency room, an operating room, and the like. The hospital bed 2320 or other form of patient support apparatus could be used in various locations without departing from the scope of the present technology.

The hospital bed 2320 comprises a controller 2330, similar to the controller 180 of hospital bed 100. The controller 2330 comprises one or more processing unit(s), one or more memories, one or more transceivers, input/output interfaces and communication interfaces (not numbered).

In one or more implementations, the controller 2330 is configured to execute one or more ML models 2335. In one or more implementations, the one or more ML models 2335 may be executed by another computing device. The one or more ML models 2335 are configured to determine a mobility index of a patient using data from the sensing device 2000, as will be explained in more detail herein below.

In some embodiments, the hospital bed 2320 may communicate with a headwall (not shown) located in a room. The headwall and hospital bed 2320 may be configured to receive transmit and/or receive data. Additionally, or alternatively, the hospital bed 2320 may connect to a hospital network, a nurse call interface, and other devices via a wired or wireless communication link with the headwall.

The sensing device 2000, of which different embodiments have been described previously, is associated with the hospital bed 2320. The sensing device 2000 may be located in proximity of the hospital bed and within a communication range therefrom. In some implementations, the sensing device 2000 may be secured to the hospital bed 2320 using various methods and attachment or connection means. In one or more other implementations, the sensing device 2000 is integrated into the hospital bed 2320, such as within a panel of the hospital bed 2320.

The sensing device 2000 may be configured to communicate with one or more of the controller 2330 of the hospital bed 2320, the headwall (not shown), a nurse station computer 2360, and the server 2370 via a respective communication link (not numbered).

The nurse station computer 2360 is a centralized computing system configured to manage and access patient information, coordinate care, and to enable and facilitate communication among healthcare professionals. The nurse station computer 2360 may be configured to store electronic medical records (EMRs), scheduling and tracking patient appointments, integrating with hospital-wide communication and monitoring systems, assisting in medication management, and providing tools for reporting and analytics.

In some embodiments of the present technology, the nurse station computer 2360 is configured to inter alia: (i) receive vital sign data and/or mobility data of a patient having been acquired by the sensing device 2000; (ii) display the vital sign data and/or mobility data; and (iii) generate and transmit information based on the vital sign data and/or mobility data.

It will be appreciated that the nurse station computer 2360 may receive the vital sign data and/or mobility data directly or indirectly from the sensing device 2000 (e.g., via the hospital bed 2320).

Additionally, the environment and system 2300 may comprise one or more medical devices and/or computing devices (e.g., mobile device such as a phone, tablet, etc.) connected to the communication network 2380 or directly to components of the environment and system 2300.

The server 2370 is configured to inter alia: (i) receive data from and transmit data to the hospital bed 2320; (ii) receive data from and transmit data to the contactless sensing device 2000, (iii) receive data from and transmit data to the nurse station computer via the communication network; (iv) receive data from and transmit data client devices; and (v) train and provide access to the one or more ML models 2335.

In some embodiments of the present technology, the server 2370 may be connected to the communication network 2380 via a communication link 2385. In alternative embodiments of the present technology, the server 2370 may be optional.

The implementation of the server 2370 is well known to the person skilled in the art of the present technology. However, briefly speaking, the server may be implemented as a computing device and comprise, one or more processors (e.g., central processing unit (CPU) and/or graphics processing unit (GPU)), a memory and/or storage unit, input/output interfaces and communication interfaces.

It will be appreciated that the server 2370 may provide the output of one or more processing steps to another electronic device for display, confirmation and/or troubleshooting. As a non-limiting example, the server 2370 may transmit data including calculated values, results, and machine learning parameters, for display on a computing device such as a smart phone, tablet, and the like.

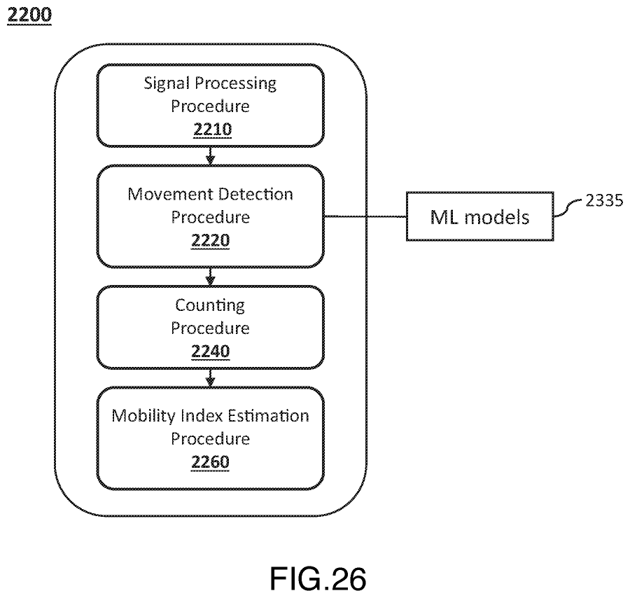

With reference to FIG. 26, a patient mobility determination procedure 2200 will now be described in accordance with one or more non-limiting embodiments of the present technology.

During the patient mobility determination procedure 2200, the sensing device 2000 is configured to generate a movement index indicative of a mobility of the patient. The movement index corresponds to a value greater than or equal to zero for a given period of time.

In one or more implementations, the patient mobility determination procedure 2200 is executed upon detection of a movement of the patient using the sensing device 2000. Detection of a movement of the patient may be correlated with interruption in the detection of vital signs by the sensing device 2000. It will be appreciated that accurate detection of vital signs with the sensing device 2000 require the patient to be stationary or almost stationary in the patient support apparatus, as movements of the patient cause variations in the received electromagnetic signals, which in turn causes interruption in detection of vital signs of the patient.

In one or more other implementations, the detection of the movements of the patient may also be initiated via other sensors associated with the patient support apparatus (e.g., load cells, pressure sensor of a mattress) detecting movement of the patient.

The movement index is used to estimate a mobility index of the patient. In one or more implementation, the mobility index is between 1 and 4, according to the Braden scale standard designed to assess the mobility of a patient supported by a patient support apparatus.

The Braden scale is a clinical tool known in the art for assessing the risks of patients developing pressure ulcers in bedridden patients. The Braden scale consists of six subscales, one of which is specifically focused on patient mobility. Each subscale is rated from 1 to 4, and an overall score is then calculated to evaluate the overall risk of pressure ulcers.

By using the sensing device 2000 to collect data via the movement index, one or more implementations of the present technology enable one or more ML models 2335 to analyze real-time information on the patient's mobility. The one or more ML models 2335 process the data collected by the sensing device 2000, compare the data to the Braden scale standard, and assign an estimated mobility level to the patient, the mobility level being between 1 and 4. The patient mobility is estimated using the patient mobility determination procedure 2200, which will now be described.

In the context of the present technology, the movement index is a measure used to assess physical disturbances using electromagnetic signals transmitted and received by the sensing device 2000. The movement index quantifies movements or changes occurring in a given environment by analyzing variations in received electromagnetic signals.

The patient mobility determination procedure 2200 comprises a signal processing procedure 2210, a movement detection procedure 2220 using one or more ML models 2235, a counting procedure 2240 and a mobility index estimation procedure 2260.

In one or more implementations, the signal processing procedure 2210 is executed by the sensing device 2000, and the movement detection procedure 2220, the counting procedure 2240 and the mobility index estimation procedure 2260 are executed by at least one processing unit associated with the hospital bed 1800, such as the controller 180.

In one or more alternative implementations, the signal processing procedure 2210, the movement detection procedure 2220, the counting procedure 2240 and the mobility index estimation procedure 2260 are executed by the same processing unit, such as the processing unit of the hospital bed 1800 or the processing unit of the sensing device 2000.

The signal processing procedure 2210 is configured to receive, from the sensing device 2000, inter alia: (i) a sample of movement indexes during a period of time; (ii) maximal amplitude of the movement indexes; and (iii) a sum of the movement indexes during the period of time.

As a non-limiting example, the movement index may be sampled at the sampling interval of the sensing device 200 to obtain 100 different values, and the signal processing procedure 2210 may receive a vector of size 102, where 100 elements correspond to values of the movement index, one element corresponds to the maximal value of the movement index, and one element corresponds to the sum of the movement indexes.

In one or more implementations, the period of time during which the movement index is determined corresponds to the period of time between which detection of vital signs by the sensing device 2000 stops (t0) and detection of vital signs restarts (tf).

The sensor device 2000 is configured to determine the movement index by comparing adjacent pulse signals transmitted and received by the sensing device 2000. Specifically, the difference between the values of consecutive pulse signals is determined according to the following pseudoformula:

Movement Index=SUM(ABSOLUTE(Signal_1−Signal_2))

where the absolute value of the difference between “Signal_1” and “Signal_2” (representing two consecutive pulse signals) is taken for all instances when these signals are received, and then the sum of these differences is calculated by the sensing device 2000 and received by the signal processing procedure 2210. In some implementations, the signal processing procedure 2210 is configured to determine the movement index.

The movement index is representative of the magnitude of change between the consecutive pulse signals. Thus, when the movement is more significant, the differences between consecutive pulse signals become more pronounced, leading to a higher value of the movement index.

The movement index is determined by self-comparison in time. That is, rather than comparing the movement index for different individuals, trends in the movement index in time are determined for the same individual. By tracking the time-series movement index for the specific individual, the historical amount of movement can be analyzed.

It should be noted that the calculation of the movement index is not solely influenced by the level of movement. Other variables may also affect the movement index. These variables include, as a non-limiting example, dimensions of the target generating the movement, the distance between the sensor and the target, as well as the angle at which the target is detected relative to the sensing device 2000.

The movement detection procedure 2220 has access to one or more trained ML models 2235.

The one or more ML models 2235 are configured to determine, based on one or more of: the sample of the movement index during the period of time, the maximal amplitude of the movement index and the sum of the movement index during the period of time, if the movement corresponds to one of a small movement and a large movement.

The one or more ML models 2235 have been trained to detect whether the movement made by the patient is a large movement or a small movement, using the movement index generated by the sensing device 2000 as an input. A small movement corresponds to movements without mobilization of the spine, while large movements involve mobilization of the spine.

In one or more implementations, the one or more ML models 2235 are implemented as classification ML models, also referred to as classifiers. The one or more ML models 2235 have been trained on annotated movement index data output from the signal processing procedure 2210. It will be appreciated that the movement index data may have been labeled by assessors (e.g., medical personnel) observing a given patient for which the movement index has been generated for a given period. In one or more implementations, the label may be one of a small movement and a large movement. It is contemplated that in alternative implementations, the label may be multiclass (three or more types of movements).

As a non-limiting example, the one or more ML models 2235 may be implemented as classification models such as, but not limited to deep neural networks, support vector machines (SVMs), decision trees, random forest, naive Bayes, logistic regression, as well as ensemble methods (e.g., AdaBoost, gradient boosting, and bagging).

In one or more implementations, the one or more ML models 2235 are implemented using the TensorFlow Lite software library. In such implementations, the one or more ML models 2235 may be executed, as a non-limiting example, by a microcontroller (e.g., implemented by the controller 180).

The one or more ML models 2235 output the classification of the movement based on the movement index, the classification being one of a small movement and a large movement of the body of the patient.

Once the one or more ML models detects a small or large movement based on the movement index, the counting procedure 2240 executes a counter to calculate the number of small movements and large movements made by the patient over a given period.

The counting procedure 2240 is configured to record the number of small movements and the number of major movements detected by the one or more ML models 2235.

The counting procedure 2240 stores, for each patient, the number of small movements and the number of large movements during a period of time.

Once the information on the number of small and large movements is collected and analyzed by counting procedure 2240, an empirical model is used to determine the patient's mobility index.

The mobility index estimation procedure 2260 is configured to determine, for each patient, based on the number of small movements and large movements over a period of time, a mobility index. The mobility index estimation procedure 2260 implements an empirical model to determine the mobility index. In some embodiments, the mobility index estimation procedure 2260 determines the mobility index based on a plurality of thresholds of small movements and large movements.

In one or more implementations, the mobility index is a value ranging from 1 to 4 following the Braden scale.

The patient mobility determination procedure 2200 is configured to transmit an indication of the mobility index. In one or more implementations, the patient mobility determination procedure 2200 procedure transmits the mobility index to a computing device such as the nurse station computer 2360. As a non-limiting example, the computing device may be a server, a mobile device, a desktop computer at a nursing station.

The mobility index may be associated with an identification of the bed, zone, room and/or patient identifier.

The mobility index enables assessing the patient's mobility.

In one or more implementations, the mobility index is transmitted with one or more vital signs of the patient. The one or more vital signs of the patient include one or more of the heart rate (HR) and respiratory rate (RR) during the given period of time.

It will be appreciated that by using the mobility index generated by the sensing device 2000, the patient mobility determination procedure 2200 provides real-time information on the patient's mobility.

It will be appreciated that the present configuration enables improving patient care as it allows continuous evaluation of the patient's mobility without requiring constant monitoring by medical staff. The use of one or more ML model(s) 2235 in combination with the data sensed by the sensing device 2000 using the patient mobility determination procedure 330 provides an automated and objective solution to assess patient mobility, facilitating clinical decision-making and tailoring care based on the detected level of mobility.

In the specific case of installing the sensing device 2000 behind a fixed location, such as a mattress or backrest of the hospital bed, the sensing device 2000 provides a more reliable and conditioned data collection. The patient mobility determination procedure 2200 could also enable the analysis of movements from different individuals, given that the sensing device 2000 is placed in a fixed location, and measurement conditions are more controlled.

Furthermore, the patient mobility determination procedure 2200 provides real-time analysis capability and enables detecting changes in patient mobility promptly, which also enables early intervention in case of deteriorating mobility of the patient, thereby contributing to the prevention of complications related to prolonged immobility, in addition to deterioration of the vital signs of the patient.

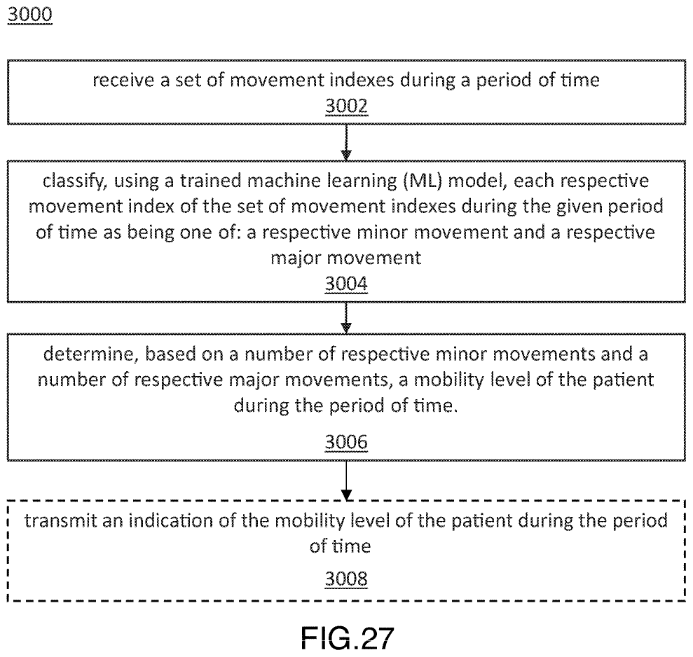

With reference to FIG. 27, a flow chart of a method 3000 of determining a mobility level of a patient will now be described in accordance with one or more non-limiting implementations of the present technology.

The method 3000 is executed by at least one processing unit. In some implementations, the method 3000 may be executed by at least one processing unit of a contactless sensing device, such as sensing devices 200, 324, 424, 524, 624, 724, 824, 924, 1024, 1124, 1224, 1324, 1424, 1524, 1624, 1724, 2000.

In other implementations, the method 3000 is executed by at least one processing unit of the contactless sensing device and the at least one processing unit of the hospital bed 100. In one or more implementations, the one or more ML models 2335 have been previously trained using the patient mobility determination procedure 2200.

Prior to processing step 3002, the contactless sensor is configured to detect vital signs of the given patient in the patient support apparatus.

According to processing step 3002, the at least one processing unit receives, from the contactless sensor, a set of movement indexes during a period of time.

The set of movement indexes are detected and calculated by the contactless sensor. The set of movement indexes comprises at least one movement index detected during a period of time. In one or more implementations, each movement index of the set of movement indexes is detected and determined by the contactless sensor based on consecutive pulse signals transmitted and received by the contactless sensor.

In one or more implementations, the period of time during which the movement index is determined corresponds to the period of time between which detection of vital signs by the sensing device 2000 stops (t0) and detection of vital signs restarts (tf).

In one or more implementations, the at least one processing unit receives and/or determines a maximal amplitude of the set of movement indexes, and a sum of the movement indexes during the period of time.

According to processing step 3004, the at least one processing unit classifies, using a trained machine learning (ML) model, each respective movement index of the set of movement indexes during the given period of time as being one of: a respective minor movement and a respective major movement.

The at least one processing unit provides the set of movement indexes, the maximal amplitude and the sum of the movement indexes as an input to the at least one trained ML model.

In one or more implementations, the at least one trained ML model has been trained to classify movements based on the mobility index detected by the contactless sensor.

According to processing step 3006, the at least one processing unit determines, based on a number of respective minor movements and a number of respective major movements, a mobility level of the patient during the period of time.

In one or more implementations, the at least one processing unit counts the number of small movements and large movements output by the at least one ML model. The at least one processing unit uses an empirical model to determine, based on the number of respective minor movements and respective major movements, the mobility level of the patient.

In one or more implementations, the mobility level of the patient is based on a Braden scale of 1 to 4.

In one or more implementations, according to processing step 3008, the at least one processing unit transmits an indication of the mobility level of the patient to another computing device. It will be appreciated that processing step 3008 may be optional.

The indication of the transmission of the mobility level of the patient may cause a notification on the computing device and/or a control panel of a patient support apparatus (e.g., control panel 106, 114 of bed 100 of FIG. 1). In one or more implementations, the indication of the mobility level may be displayed with an indication of the period of time. It will be appreciated that the notification comprising an indication of the mobility level may be visual, audio, haptic and or multimodal.

The method 3000 ends.

The embodiments described above are intended to be examples only.

1. A system for monitoring at least one vital sign of a patient, the system comprising:
a patient support apparatus comprising:
a patient platform comprising:
a plurality of body panels including a backrest for supporting at least a patient's torso;
a frame, the patient platform being mounted to the frame; and
at least one contactless sensing device configured to be positioned in proximity to the patient support apparatus to detect at least one vital sign within a respective predetermined field of detection, the at least one contactless sensing device being configured to:
transmit electromagnetic signals towards a portion of the body of the patient;
receive reflected electromagnetic signals; and
detect, based on the transmitted and received reflected electromagnetic signals, at least one vital sign of the patient irrespective of: the patient's anthropomorphic measurements, a patient position within the patient support apparatus, a configuration of the patient support apparatus, and a location of the patient support apparatus.

2. The system of claim 1, wherein the at least one contactless sensing device is mounted to a respective adjustable positioning mechanism configured to adjust the at least one contactless sensing device to include the patient within the field of detection.

3. The system of claim 2, wherein the respective adjustable positioning mechanism is configured to adjust the at least one contactless sensing device by performing at least one of:
pivoting the at least one contactless sensing device about at least one pivot axis, and
positioning the at least one contactless sensing device along at least one translation axis.

4. The system of claim 3, further comprising a further sensor configured to estimate a patient position; and wherein the respective adjustable positioning mechanism is operatively connected to the further sensor to adjust the plurality of sensing devices based on the patient position.

5. The system of claim 4, wherein the further sensor comprises at least one of: load cells of the patient support apparatus, pressure sensors of a mattress, and a camera.

6. The system of any one of claims 1 to 5, wherein the at least one contactless sensing device is one of:
integrated within the backrest of the patient support apparatus, and
mounted to an underside of the backrest of the patient support apparatus.

7. The system of claim 6, wherein the at least one contactless sensing device is a single contactless sensing device comprising a sensor oriented at a respective angle towards a head end of the patient support apparatus, the respective angle being based on a field of view of the sensor to enlarge the field of detection.

8. The system of claim 7, wherein the respective angle corresponds to 90 degrees minus the field of view of the sensor in degrees.

9. The system of any one of claims 1 to 5, wherein the at least one contactless sensing device is one of:
integrated within a respective siderail of the patient support apparatus, and
mounted to a respective siderail of the patient support apparatus.

10. The system of any one of claims 1 to 5, wherein the at least one contactless sensing device is one of:
integrated within a headboard of the patient support apparatus, and
mounted to a headboard of the patient support apparatus.

11. The system of any one of claims 1 to 5, wherein the at least one contactless sensing device is one of:
integrated within a footboard of the patient support apparatus, and
mounted to a footboard of the patient support apparatus.

12. The system of any one of claims 1 to 5, wherein the at least one contactless sensing device is mounted above the patient platform on a vertical support secured to the patient support apparatus.

13. The system of any one of claims 1 to 5, wherein the at least one contactless sensing device is mounted to one of a ceiling and a wall.

14. The system of any one of claims 1 to 13, wherein the at least one contactless sensing device comprises a plurality of contactless sensing devices, each of the plurality of contactless sensing devices being positioned at a respective position, the plurality of contactless sensing devices having non-overlapping respective fields of view.

15. The system of claim 14, wherein the plurality of contactless sensing devices comprise a first contactless sensing device coupled to a first side of the patient support apparatus and a second contactless sensing device coupled to a second side opposite the first side of the patient support apparatus.

16. The system of any one of claims 1 to 15, wherein the at least one contactless sensing device is configured to use radiofrequency (RF) ranges.

17. The system of claims 16, wherein the at least one contactless sensing device is configured to use one of: millimeter waves (mmWave) frequencies, ultra-wideband (UWB) frequencies, and Wi-Fi frequencies.

18. The system of claim 17, wherein the field of detection of the at least one contactless sensing device has a maximum detection range of at least 1.5 meters.

19. The system of any one of claims 1 to 18, wherein the at least one vital sign of the patient comprises at least one of: a heart rate and a respiratory rate.

20. The system of any one of claims 1 to 19, wherein the at least one contactless sensing device is configured to detect movements of the patient based on the transmitted and received reflected electromagnetic signals.

21. The system of claim 20, wherein the at least one contactless sensing device is configured to detect movements of the patient when the at least one contactless sensing device does not detect the at least one vital sign.

22. The system of claim 20 or 21, wherein the contactless sensing device is configured to determine a movement index indicative of a level of motion of the patient supported by the patient platform.

23. The system of claim 22, wherein a processing unit is configured to:
receive a set of movement indexes for a given period of time;
classify, using a trained machine learning model, each respective movement index of the set of movement indexes during the given period of time as being one of: a respective minor movement and a respective major movement; and
determine, based on a number of respective minor movements and a number of respective major movements, a mobility level of the patient during the period of time.

24. The system of claim 23, wherein the processing unit is configured to use an empirical model to determine the mobility level of the patient during the period of time.

25. The system of claim 23 or 24, wherein the mobility level is based on a Braden scale.

26. The system of claim 1, wherein the at least one contactless sensing device is configured use optical frequencies.

27. The system of claim 1, wherein
the at least one contactless sensing device is configured to use infrared frequencies, and
wherein
the at least one vital sign comprises a body temperature of the patient.

28. A patient support apparatus comprising:
a patient platform comprising:
a plurality of body panels including a backrest for supporting at least a patient's torso;
a frame, the patient platform being mounted to the frame; and
at least one contactless sensing device configured to be coupled to the patient support apparatus to detect at least one vital sign within a predetermined field of detection, the at least one contactless sensing device being configured to:
transmit electromagnetic signals towards a portion of the body of the patient;
receive reflected electromagnetic signals; and
detect, based on the transmitted and received reflected electromagnetic signals, at least one vital sign of the patient within the predetermined field of detection, irrespective of:
anthropomorphic measurements of the patient, a patient position within the patient support apparatus, a configuration of the patient support apparatus, and a location of the patient support apparatus.

29. The patient support apparatus of claim 28, wherein the at least one contactless sensing device configured to be coupled to the patient support apparatus by being one of: being mounted on respective component of the patient support apparatus, and integrated to a respective component of the patient support apparatus.

30. The patient support apparatus of claim 29, wherein the at least one contactless sensing device is mounted to a respective adjustable positioning mechanism configured to adjust the at least one contactless sensing device to include a portion of a body of the patient within the field of detection, the respective adjustable positioning mechanism being coupled to the respective component of the patient support apparatus.

31. The patient support apparatus of claim 30, wherein the respective adjustable positioning mechanism is configured to adjust the at least one contactless sensing device by performing at least one of:
pivoting the at least one contactless sensing device about at least one pivot axis, and
positioning the at least one contactless sensing device along at least one translation axis.

32. The patient support apparatus of any one of the claims 28 to 31, wherein
the patient support apparatus comprises a headboard, a footboard and siderails; and wherein
the respective component of the patient support apparatus comprises at least one of headboard, a footboard and siderails.

33. The patient support apparatus of any one of claims 28 or 29, wherein the at least one contactless sensing device is coupled to an underside of the backrest.

34. The patient support apparatus of claim 33, wherein the contactless sensing device is positioned and oriented towards the patient's lower back.

35. The patient support apparatus of claim 33, wherein the at least one contactless sensing device is a single contactless sensing device comprising a sensor oriented at a respective angle towards a head end of the patient support apparatus, the respective angle being based on a field of view of the sensor to enlarge the field of detection.

36. The patient support apparatus of claim 35, wherein the respective angle corresponds to 90 minus the field of view of the sensing device in degrees.

37. The patient support apparatus of any one of claims 28 to 32, wherein the at least one contactless sensing device comprises a plurality of contactless sensing devices, each of the plurality of contactless sensing devices being positioned at a respective position, the plurality of contactless sensing devices having non-overlapping respective fields of detection within the patient platform of the patient support apparatus.

38. The patient support apparatus of claim 37, wherein the plurality of contactless sensing devices comprise a first contactless sensing device coupled to a first side of the patient support apparatus and a second contactless sensing device coupled to a second side opposite the first side of the patient support apparatus.

39. The patient support apparatus of any of claims 28 to 38, wherein the at least one contactless sensing device is configured to use radiofrequency (RF) ranges.

40. The patient support apparatus of claim 39, wherein the at least one contactless sensing device is configured to use one of: millimeter waves (mmWave), ultra-wideband (UWB) and Wi-Fi frequencies.

41. The patient support apparatus of claim 40, wherein the field of detection of the at least one contactless sensing device has a maximum detection range of at least 1.5 m.

42. The patient support apparatus of any one of claims 28 to 41, wherein the at least one vital sign of the patient comprises at least one of: a heart rate and a respiratory rate.

43. The patient support apparatus of any one of claims 28 to 43, wherein the at least one contactless sensing device is configured to detect movements of the patient based on the transmitted and received reflected electromagnetic signals.

44. The patient support apparatus of claim 43, wherein the at least one contactless sensing device is configured to detect movements of the patient when the at least one contactless sensing device does not detect the at least one vital sign.

45. The patient support apparatus of claim 44, wherein the contactless sensing device is configured to determine a movement index indicative of a level of motion of the patient supported by the patient platform.

46. The patient support apparatus of claim 45, wherein a processing unit is configured to:
receive a set of movement indexes for a given period of time;
classify, using a trained machine learning model, each respective movement index of the set of movement indexes during the given period of time as being one of: a respective minor movement and a respective major movement; and
determine, based on a number of respective minor movements and a number of respective major movements, a mobility level of the patient during the period of time.

47. The patient support apparatus of claim 46, wherein the processing unit is configured to use an empirical model to determine the mobility level of the patient during the period of time.

48. The patient support apparatus of claim 46 or 47, wherein the mobility level is based on a Braden scale.

49. The patient support apparatus of any one of claim 28, wherein the at least one contactless sensing device is configured to use optical frequencies.

50. A method for determining a mobility of a patient supported a patient support apparatus, the patient support apparatus being associated with a contactless sensing device configured to detect vital signs of a patient, the method being executed by at least one processing unit, the method comprising:
receiving, from the sensing device, a set of movement indexes indicative of a movement of the patient during a period of time;
classifying, using a trained machine learning model, each respective movement index of the set of movement indexes during the period of time as being one of: a respective minor movement and a respective major movement; and
determining, based on a respective number of minor movements and a respective number of major movements, a mobility level of the patient during the period of time.

51. The method of claim 50,
further comprising:
receiving a maximal amplitude of the set of movement indexes and a sum of the movement indexes; and wherein
said classifying, using the trained machine learning model, each respective movement index of the set of movement indexes during the period of time as being one of: the respective minor movement and the respective major movement is further based on the maximal amplitude of the set of movement indexes and a sum of the movement indexes.

52. The method of claim 50 or 51, wherein the at least one processing unit is configured to use an empirical model to determine the mobility level of the patient during the period of time.

53. The method of claim 52, wherein the mobility level is based on a Braden scale.

54. The method of any one of claims 50 to 53, further comprising: detecting, by the contactless sensing device, before the given period of time, at least one vital sign of the patient.

55. The method of any one of claims 50 to 54, wherein the at least one vital sign comprises at least one of a heart rate (HR) and a respiratory rate (RR).

56. The method of any one of claims 50 to 55, further comprising: transmitting an indication of the mobility level of the patient to a computing device of a medical professional.
